SEQUENCE LISTING (41 sequences)

SEQ ID NO: 1 - protein, 122 aa, synthetic construct [sequence omitted]

SEQ ID NO: 2 - protein, 107 aa, synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 3 - protein, 107 aa, synthetic construct
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIK

SEQ ID NO: 4 - protein, 451 aa, synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 448 aa, synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 214 aa, synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, synthetic construct
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1356 bp, synthetic construct
  source 1..1356: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 9 - DNA, 1347 bp, synthetic construct
  source 1..1347: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 10 - DNA, 645 bp, synthetic construct
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttactggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - DNA, 645 bp, synthetic construct
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttagtggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 5 aa, synthetic construct
RYYMY

SEQ ID NO: 13 - protein, 17 aa, synthetic construct
EINPNNGGTKFNEKFKN

SEQ ID NO: 14 - protein, 13 aa, synthetic construct
EDDYDGTPFAMDY

SEQ ID NO: 15 - protein, 11 aa, synthetic construct
HASQNINVWLS

SEQ ID NO: 16 - protein, 7 aa, synthetic construct
KASNLHT

SEQ ID NO: 17 - protein, 9 aa, synthetic construct
QQGQTYPYT

SEQ ID NO: 18 - protein, 5 aa, synthetic construct
RYYLY

SEQ ID NO: 19 - protein, 17 aa, synthetic construct
EINPNNGGTKFNEKFKS

SEQ ID NO: 20 - protein, 13 aa, synthetic construct
EDDYDGTPDAMDY

SEQ ID NO: 21 - protein, 11 aa, synthetic construct
HASQNINLWLS

SEQ ID NO: 22 - protein, 7 aa, synthetic construct
RASNLHT

SEQ ID NO: 23 - protein, 9 aa, synthetic construct
QQGHSYPYT

SEQ ID NO: 24 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 25 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 26 - protein, 584 aa, synthetic construct
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 27 - protein, 237 aa, Macaca fascicularis
MGSSLAFLLLSFHVCVLLLQLLMPHSAQFAVVGPPGPILAMVGEDADLPCHLFPTMSAETMELRWVSSNLRQVVNVYADGKEVEDRQSAAYRGRTSILRDGITAGKAALRIHNVTASDSGKYLCYFQDGDFYEKALVELKVAALGSDLHIDVKGYEDGGIHLECRSTGWYPQPQIRWSNDKGENIPAVEAPVFVDGVGLYAVAASVILRGSSGEGVSCTIRSSLLGLEKTTSISIAG

SEQ ID NO: 28 - protein, 237 aa, Macaca fascicularis
MGSSLAFLLLNFHVSFFLVQLLTPCSAQFSVLGPSGPILAMVGEDADLPCHLFPTMSAETMELRWVSSSLRQVVNVYADGKEVEDRQSAPYRGRTSILRDDIAAGKAALRIHNVTASDSGKYLCYFQDADFYEKALVELKVAALGSNLHVEVKGYEDGGIHLECRSTGWYPQPKIQWSNAKGQNIPAVEAPVVADGVGLYAVAASVIMRGGSGESVSCIIRNSVLGLEKTASISIAD

SEQ ID NO: 29 - protein, 237 aa, Macaca fascicularis
MANFLAFLLLNFRVCLLLVQLLTPCSAQFAVLGPHGPILAMVGEDVDLPCHLFPTMSAETMELRWVSSSLRQVVNVYSDGKEVEDRQSAPYRGRTSILRDGITAGKAALRIHNVTASDSGKYLCYFQDGDFYEKALVELKVAALGSDLHIEVKGYEDGGIHLECRSTGWYPQPQIQWSNTKGQHIPAVKAPVVADGVGLYAVAASVIMRGSSGEGVSCIIRNSLLGLEKTASISITD

SEQ ID NO: 30 - protein, 122 aa, synthetic construct
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSS

SEQ ID NO: 31 - protein, 107 aa, synthetic construct
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIK

SEQ ID NO: 32 - protein, 452 aa, synthetic construct
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, synthetic construct
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 5 aa, synthetic construct
RYYLY

SEQ ID NO: 35 - protein, 17 aa, synthetic construct
EINPSNGGTNFNEKFKS

SEQ ID NO: 36 - protein, 13 aa, synthetic construct
EDDYDGTPDAMDY

SEQ ID NO: 37 - protein, 11 aa, synthetic construct
HASQNINVWLS

SEQ ID NO: 38 - protein, 7 aa, synthetic construct
KASNLHT

SEQ ID NO: 39 - protein, 8 aa, synthetic construct
QGQSYPLT

SEQ ID NO: 40 - protein, 122 aa, synthetic construct
QVQLQQSGAELVKPGASVKLSCKASGYTFTRYYLYWVKQRPGQGLEWIGEINPSNGGTNFNEKFKSKATLTVDKSSRTTYIQLSSLTSEDSAVYYCSREDDYDGTPDAMDYWGQGTAVTVSS

SEQ ID NO: 41 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGGGTKLDIK

(19) United States
(12) Patent Application Publication
(10) Pub. No.: US 2026/0256909 A1
(43) Pub. Date: Sep. 3, 2026

(54) COMBINATION OF A BTN3A ACTIVATING ANTIBODY, A BCL2 INHIBITOR AND HYPOMETHYLATING AGENT FOR USE IN TREATING CANCER

(71) Applicants: Imcheck Therapeutics, Marseille (FR); INSTITUT NATIONAL DE LA SANTÉ ET DE LA RECHERCHE MÉDICALE, PARIS (FR); CENTRE NATIONAL DE LA RECHERCHE SCIENTIFIQUE, Paris (FR); UNIVERSITÉ D'AIX-MARSEILLE, MARSEILLE (FR); INSTITUT JEAN PAOLI & IRENE CALMETTES, Marseille (FR)

(72) Inventors: Anne-Charlotte LE FLOCH, Marseille (FR); Daniel OLIVE, Marseille (FR); Norbert VEY, Marseille (FR); Paul FROHNA, Marseille (FR); Elisabeth WIEDUWILD, Marseille (FR); Loui MAKADAMUTIL, Del Mar, CA (US)

(21) Appl. No.: 19/118,694

(22) PCT Filed: Oct. 3, 2023
(86) PCT No.: PCT/EP2023/077342

§ 371 Date: Apr. 4, 2025

(30) Foreign Application Priority Data
Oct. 4, 2022 (EP) 22306469.2
Jul. 18, 2023 (EP) 23186069.3

(51) Int. Cl. A61K 39/395; A61K 31/635; A61K 31/706; A61K 39/00; A61P 35/00

(52) U.S. Cl. CPC A61K 39/3955; A61K 31/635; A61K 31/706; A61P 35/00; A61K 2039/505; A61K 2039/54; A61K 2039/55

(57) ABSTRACT

The present invention relates a therapeutic combination of a BTN3A activating antibody, a Bcl-2 family inhibitor and hypomethylating agents that is particularly useful for the treatment of cancer, in particular hematological malignancies. The present disclosure more particularly relates to the combined use of a BTN3A activating antibody that activates the cytolytic function of Vγ9Vδ2 T cells, and of Venetoclax, which selectively inhibits the Bcl2 receptor, and hypomethylating agents such as Azacytidine, to promote synergistically and specifically Vγ9Vδ2 T cell anticancer activity.

### CROSS REFERENCE TO RELATED APPLICATIONS

This application is a National Stage Entry under 35 U.S.C. § 371 of International Application No. PCT/EP2023/077342 filed Oct. 3, 2023, which claims the benefit and priority of European Application No. 23186069.3 filed Jul. 18, 2023, and European Application No. 22306469.2 filed on Oct. 4, 2022, the contents of each of which are incorporated herein by reference.

### REFERENCE TO A SEQUENCE LISTING

This application contains a Sequence Listing as a separate part of the disclosure. The contents of the Sequence Listing (2025-04-04-IMP-03-US-Sequence Listing.xml; Size: 45,332 bytes; and Date of Creation: Apr. 1, 2025) are incorporated herein by reference.

It is hereafter disclosed a therapeutic combination of a BTN3A activating antibody, a Bcl-2 inhibitor and hypomethylating agents that is particularly useful for the treatment of cancer, in particular hematological malignancies. The present disclosure more particularly relates to the combined use of a BTN3A activating antibody that activates the cytolytic function of Vγ9Vδ2 T cells, and of Venetoclax, which selectively inhibits the Bcl2 receptor, and hypomethylating agents such as Azacytidine, to promote synergistically and specifically Vγ9Vδ2 T cell anticancer activity.

### BACKGROUND

To date, various therapeutic and vaccine strategies have been proposed for the treatment of cancer that rely on a mobilization of patient immune responses, and more specifically T lymphocytes, against malignant cells. Several immunomodulatory antibodies to CTLA-4, PD-1 and PD-L1 have already been approved for clinical use by multiple regulatory agencies throughout the world. Although these drugs represent major advances in cancer therapy, there remain unmet medical needs for large parts of cancer patient populations that do not respond to the currently available treatments.

Gamma delta (γδ) T cells are an unconventional T cell subset with both innate and adaptive immune response features that play a key role in the immunosurveillance against malignancies and infections (Holtmeier W, et al, editors. Chem Immunol Allergy [Internet]. Basel: KARGER; 2005 [cited 2022 Aug. 19]. p. 151-183. Available from: https://www.karger.com/Article/FullText/86659). In the blood of healthy human adults, Vγ9Vδ2 T cells constitute the majority of circulating γδ T cells (50 to 90%) (Kabelitz D, et al. Cell Mol Immunol. 2020 September; 17(9):925-939) and represent 1 to 5% of total blood lymphocytes (Pauza C D, et al. Gamma Delta T Cell Therapy for Cancer: It Is Good to be Local. Front Immunol [Internet]. 2018 [cited 2018 Nov. 19]). Unlike conventional as T cells that represent the major T cell subset among circulating T cells and recognize MHC-restricted antigens, γδ T cells are activated in a non-MHC-restricted manner. More specifically, Vγ9Vδ2 T cell activation is triggered by the intracellular accumulation of phosphoantigens (pAg) overproduced in response to viral and bacterial infections, metabolic stress, or genetic dysregulation during carcinogenesis. Vγ9Vδ2 T cell activation under these pathophysiologic contexts induces broad functional activities that include the production of cytokines and chemokines, the cytolysis of infected or transformed target cells, and interactions with other cells including epithelial cells, monocytes, dendritic cells (DC), neutrophils, and B cells (Blazquez J L, Benyamine A, Pasero C, Olive D. New Insights Into the Regulation of γδ T Cells by BTN3A and Other BTN/BTNL in Tumor Immunity. Front Immunol [Internet]. 2018 [cited 2018 Aug. 6]). With their potent anti-tumor activity (Pauza C D et al., see supra) and the association between their infiltration into malignant tissue and positive prognosis (Gentles A J, et al. Nat Med. 2015 August; 21(8):938-945; Tosolini M, et al. OncoImmunology. 2017 Mar. 4; 6(3):e1284723. PMID: 28405516), Vγ9Vδ2 T cells represent an attractive target for cancer immunotherapy.

A number of Vγ9Vδ2 T cell-based immune-oncology therapeutic approaches have been explored in a variety of tumors in recent years using either in vivo activation of Vγ9Vδ2 T cells with aminobisphosphonates (ABP) such as Zoledronate or synthetic phosphoantigens (i.e. BrHPP) in combination with IL-2, or adoptive transfer of autologous or allogeneic Vγ9Vδ2 T cells to patients following in vitro/ex vivo expansion (Kabelitz D, et al. 2020, see supra). While these two Vγ9Vδ2 T cell-based therapies appear to be safe, clinical responses obtained were variable among patients (Kabelitz D, et al. 2020, see supra). Particularly in leukemia, only low responses have been observed in clinical trials with the in vivo stimulation of Vγ9Vδ2 T cells using ABP. In contrast, expansion and transfer of Vγ9Vδ2 T cells had over 50% response rates (Kunkele K P, et al. Cells. 2020 Mar. 30; 9(4):829; Barros M de S et al. Front Immunol. 2021 Sep. 22; 12:729085), strengthening the utility of Vγ9Vδ2 T cells in particular in AML therapy, and their use is further investigated in numerous clinical trials (Saura-Esteller J, et al. Front Immunol. 2022 Jun. 16; 13:915837). Though apparently successful, in vitro/ex vivo expansion and transfer of Vγ9Vδ2 T cells is laborious, suggesting that novel strategies to enhance the clinical benefit of Vγ9Vδ2 T cell-mediated anti-tumor immunity using in vivo activation and expansion are needed.

The patent publications WO2012080351A1, WO2012080769A1, WO20200257031A1, and WO2020136218 refer to various antibodies against BTN3A able to activate the cytokine production, proliferation and cytolytic function of Vγ9Vδ2 T cells.

Previous studies have shown that Venetoclax and the hypomethylating agent 5-Azacytidine have direct anti-leukemic activity. In addition, 5-Azacytidine was shown to improve cancer cell recognition by immune effector cells through induction of stress ligand expression (Gang A O, et al. Blood Cancer J. 2014 March; 4(3):e197-e197; Lee J B, et al. Blood. 2021 Jul. 22; 138(3):234-245), and Venetoclax was described to enhance T cell and NK cell-mediated cytotoxicity against AML blasts (Lee, et al. 2021, supra; Wu, et al. Int Immunopharmacol. 2022 March; 104:108497). However, Venetoclax treatment leads to lymphopenia in AML patients, which may be clinically detrimental given that Vγ9Vδ2 T cells are normally <5% of total T cells in human adult peripheral blood.

The inventors have shown that ICT01-mediated activation of Vγ9Vδ2 T cells partially protects them from Venetoclax-induced cell death and that the combination of ICT01 with Venetoclax and 5-Azacytidine significantly improves AML cell killing.

The present invention relies on the use of Venetoclax and hypomethylating agents in combination with ICT01 that demonstrates superior AML killing compared to single agents. By triggering Vγ9Vδ2 T cell anti-cancer activity, thus protecting them from cell death, and chemotherapy-induced apoptosis in AML cells.

### SUMMARY

### Specific Embodiments

E1. A BTN3A activating antibody, for use in the treatment of cancer in a subject in need thereof, wherein a therapeutically efficient amount of said BTN3A activating antibody is administered to said subject, simultaneously, sequentially or separately, with a therapeutically efficient amount of a Bcl2 family inhibitor compound, for example a Bcl2 inhibitor, optionally further in combination with a hypomethylating agent.

E2. The BTN3A activating antibody, for use according to embodiment E1, wherein said Bcl2 inhibitor is Venetoclax.

E3. The BTN3A activating antibody for use according to embodiment E1 or E2, wherein said BTN3A activating antibody binds to human BTN3A with a K_{D} of 10 nM or less, preferably with a K_{D} of 5 nM or less, as measured by surface plasmon resonance.

E4. The BTN3A activating antibody for use according to any one of embodiment E1-E3, wherein said BTN3A activating antibody induces the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay.

E5. The BTN3A activating antibody for use according to any one of embodiments E1-E4, wherein said BTN3A activating antibody:
comprises (a) a variable heavy chain (VH) polypeptide comprising an amino acid sequence that is at least about 95%, 96%, 97%, 98%, 99% or 100% identical to the amino acid sequence of SEQ ID NO:1, and (b) a variable light chain (VL) polypeptide comprising an amino acid sequence that is at least about 95%, 96%, 97%, 98%, 99% or 100% identical to of SEQ ID NO:2 or SEQ ID NO:3;
comprises HCDRs1-3 of SEQ ID NO:12-14 and LCDRs1-3 of SEQ ID NO:15-17;
comprises HCDRs1-3 of SEQ ID NO:18-20 and LCDRs1-3 of SEQ ID NO:21-23; or
competes for binding with an antibody selected from mAb 20.1 as produced by the hybridoma deposited at the CNCM under deposit number I-4401, mAb 7.2 as produced by the hybridoma deposited at the CNCM under deposit number I-4402, and an antibody having a heavy chain of SEQ ID NO:4 and a light chain of SEQ ID NO:6.

E6. The BTN3A activating antibody for use according to any one of embodiments E1-E5, wherein said BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E7. The BTN3A activating antibody for use according to any one of embodiments E1-E6, wherein said mutant IgG1 constant region is the IgG1 triple mutant L247F L248E and P350S.

E8. The BTN3A activating antibody for use according to any one of embodiment E1-E7, wherein the anti-BTN3A antibody is an antibody comprising a heavy chain of SEQ ID NO: 4 and a light chain of SEQ ID NO: 6.

E9. The BTN3A activating antibody for use according to any one of embodiments E1-E8, wherein said BTN3A activating antibody is administered in combination simultaneously, sequentially or separately with Venetoclax, further in combination with Azacytidine or Decitabine.

E10. The BTN3A activating antibody for use according to any one of embodiments E1-E9, wherein said cancer is a hematological malignancy.

E11. The anti BTN3A activating antibody for use according to any one of embodiments E1-E10, wherein said cancer is acute myeloid leukemia.

E12. The BTN3A activating antibody for use according to any one of embodiments E1-E11, wherein said cancer is acute myeloid leukemia, and wherein said BTN3A activating antibody is an antibody comprising a heavy chain of SEQ ID NO: 4 and a light chain of SEQ ID NO: 6, and said BTN3A activating antibody is administered in combination simultaneously, sequentially or separately with Venetoclax, further in combination with Azacytidine or Decitabine.

E13. The BTN3A activating antibody for use according to any one of embodiments E1-E12, wherein said subject is not eligible for intensive chemotherapy.

E14. The BTN3A activating antibody for use according to any one of embodiments E1-E13, wherein said subject is older than 75.

E15. The BTN3A activating antibody for use according to any one of embodiments E1-E14, wherein said BTN3A activating antibody is administered once every three weeks, or once every four weeks.

E16. The BTN3A activating antibody for use according to any one of embodiments E1-E15, wherein the BTN3A activating antibody is administered intravenously.

E17. The BTN3A activating antibody for use according to any one of embodiments E1-E16, wherein the BTN3A activating antibody is administered at a unit dose of about 7 to about 200 mg, e.g., about 75 mg, for example once every 21 days for 1 to 22 cycles.

E18. The BTN3A activating antibody for use according to any one of embodiments E1-E17, wherein Venetoclax is administered orally, once a day.

E19. The BTN3A activating antibody for use according to any one of embodiments E1-E18, wherein Venetoclax is administered orally at a unit dose of about 50 mg to about 500 mg.

E20. The BTN3A activating antibody for use according to any one of embodiments E1-E19, wherein Azacitidine or Decitabine is administered as the hypomethylating agent.

E21. The BTN3A activating antibody for use according to any one of embodiments E1-E20, wherein Azacitidine is administered as the hypomethylating agent.

E22. The BTN3A activating antibody for use according to any one of embodiments E1-E21, wherein a hypomethylating agent is administered at a dose of about 50 mg/m² to about 100 mg/m².

E23. The BTN3A activating antibody for use according to any one of embodiments E1-E22, wherein a hypomethylating agent is administered at a dose of about 75 mg/m².

E24. The BTN3A activating antibody for use according to any one of embodiments E1-E23, wherein a hypomethylating agent is administered once a day.

E25. The BTN3A activating antibody for use according to any one of embodiments E1-E24, wherein a hypomethylating agent is administered for 5-7 consecutive days.

E26. The BTN3A activating antibody for use according to any one of embodiments E1-E25, wherein a hypomethylating agent is administered for
(a) seven consecutive days on days 1-7 of a 28-day cycle,
(b) five consecutive days on days 1-5, followed by a two-day break, then two consecutive days on days 8-9, of a 28-day cycle, or
(c) six consecutive days on days 1-6, followed by a one-day break, then optionally one administration on day 8, of a 28-day cycle.

E27. The BTN3A activating antibody for use according to any one of embodiments E1-E26, wherein a hypomethylating agent is administered subcutaneously or intravenously.

E28. The BTN3A activating antibody for use according to any one of embodiments E1-E27, wherein Venetoclax is first administered for at least one cycle followed by a recovery period and said BTN3A activating antibody is administered for example, after at least 10 to 14 days of a recovery period, or along with cycle 2 of administration of Venetoclax.

E29. The BTN3A activating antibody for use according to any one of embodiments E1-E27, wherein the first administration of Venetoclax occurs after a first cycle of treatment with BTN2A activating antibody, preferably after 21 days following the first administration of the BTN3A activating antibody.

E30. A method of treating cancer in a subject in need thereof, said method comprising administering a therapeutically efficient amount of a BTN3A activating antibody to said subject, simultaneously, sequentially or separately, with a therapeutically efficient amount of a Bcl2 family inhibitor compound, for example a Bcle 2 inhibitor, optionally further in combination with a hypomethylating agent.

E31. The method of embodiment E30, wherein said Bcl2 inhibitor is Venetoclax.

E32. The method of embodiment E30 or E31, wherein said BTN3A activating antibody binds to human BTN3A with a K_{D} of 10 nM or less, preferably with a K_{D} of 5 nM or less, as measured by surface plasmon resonance.

E33. The method of any one of embodiments E30-E32, wherein said BTN3A activating antibody induces the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay.

E34. The method of any one of embodiments E30-E33, wherein said BTN3A activating antibody:
comprises (a) a variable heavy chain (VH) polypeptide comprising an amino acid sequence that is at least about 95%, 96%, 97%, 98%, 99% or 100% identical to the amino acid sequence of SEQ ID NO:1, and (b) a variable light chain (VL) polypeptide comprising an amino acid sequence that is at least about 95%, 96%, 97%, 98%, 99% or 100% identical to of SEQ ID NO:2 or SEQ ID NO:3;
comprises HCDRs1-3 of SEQ ID NO:12-14 and LCDRs1-3 of SEQ ID NO:15-17;
comprises HCDRs1-3 of SEQ ID NO:18-20 and LCDRs1-3 of SEQ ID NO:21-23; or
competes for binding with an antibody selected from mAb 20.1 as produced by the hybridoma deposited at the CNCM under deposit number I-4401, mAb 7.2 as produced by the hybridoma deposited at the CNCM under deposit number I-4402, and an antibody having a heavy chain of SEQ ID NO:4 and a light chain of SEQ ID NO:6.

E35. The method of any one of embodiments E30-E34, wherein said BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E36. The method of any one of embodiments E30-E35, wherein said mutant IgG1 constant region is the IgG1 triple mutant L247F L248E and P350S.

E37. The method of any one of embodiment E30-E36, wherein the anti-BTN3A antibody is an antibody comprising a heavy chain of SEQ ID NO: 4 and a light chain of SEQ ID NO: 6.

E38. The method of any one of embodiments E30-E37, wherein said BTN3A activating antibody is administered in combination simultaneously, sequentially or separately with Venetoclax, further in combination with Azacytidine or Decitabine.

E39. The method of any one of embodiments E30-E38, wherein said cancer is a hematological malignancy.

E40. The method of any one of embodiments E30-E39, wherein said cancer is acute myeloid leukemia.

E41. The method of any one of embodiments E30-E40, wherein said cancer is acute myeloid leukemia, and wherein said BTN3A activating antibody is an antibody comprising a heavy chain of SEQ ID NO: 4 and a light chain of SEQ ID NO: 6, and said BTN3A activating antibody is administered in combination simultaneously, sequentially or separately with Venetoclax, further in combination with Azacytidine or Decitabine.

E42. The method of any one of embodiments E30-E41, wherein said subject is not eligible for intensive chemotherapy.

E43. The method of any one of embodiments E30-E42, wherein said subject is older than 75.

E44. The method of any one of embodiments E30-E43, wherein said BTN3A activating antibody is administered once every three weeks, or once every four weeks.

E45. The method of any one of embodiments E30-E44, wherein the BTN3A activating antibody is administered intravenously.

E46. The method of any one of embodiments E30-E45, wherein the BTN3A activating antibody is administered at a unit dose of about 7 to about 200 mg, e.g., about 75 mg, for example once every 21 days for 1 to 22 cycles.

E47. The method of any one of embodiments E30-E46, wherein Venetoclax is administered orally, once a day.

E48. The method of any one of embodiments E30-E47, wherein Venetoclax is administered orally at a unit dose of about 50 mg to about 500 mg.

E49. The method of any one of embodiments E30-E48, wherein Azacitidine or Decitabine is administered as the hypomethylating agent.

E50. The method of any one of embodiments E30-E49, wherein Azacitidine is administered as the hypomethylating agent.

E51. The method of any one of embodiments E30-E50, wherein a hypomethylating agent is administered at a dose of about 50 mg/m² to about 100 mg/m².

E52. The method of any one of embodiments E30-E51, wherein a hypomethylating agent is administered at a dose of about 75 mg/m².

E53. The method of any one of embodiments E30-E52, wherein a hypomethylating agent is administered once a day.

E54. The method of any one of embodiments E30-E53, wherein a hypomethylating agent is administered for 5-7 consecutive days.

E55. The method of one of embodiments E30-E54, wherein a hypomethylating agent is administered for
(a) seven consecutive days on days 1-7 of a 28-day cycle,
(b) five consecutive days on days 1-5, followed by a two-day break, then two consecutive days on days 8-9, of a 28-day cycle, or
(c) six consecutive days on days 1-6, followed by a one-day break, then optionally one administration on day 8, of a 28-day cycle.

E56. The method of any one of embodiments E30-E55, wherein a hypomethylating agent is administered subcutaneously or intravenously.

E57. The method of any one of embodiments E30-E56, wherein Venetoclax is first administered for at least one cycle followed by a recovery period and said BTN3A activating antibody is administered for example, after at least 10 to 14 days of a recovery period, or along with cycle 2 of administration of Venetoclax.

E58. The method of any one of embodiments E30-E56, wherein the first administration of Venetoclax occurs after a first cycle of treatment with BTN2A activating antibody, preferably after 21 days following the first administration of the BTN3A activating antibody.

### FIGURES

FIG. 1: Activation of Vγ9Vδ2 T Cells with ICT01 Protects Them from Venetoclax Induced Cell Death. A) Mean±SEM frequencies of Live (black), Apoptotic (light grey) or Dead (dark grey) Vγ9Vδ2 T cells in HD-PBMC 48 h after treatment with Venetoclax (left) or 5-Azacytidine (right). N=4 HD. B) Mean frequency of Live Vγ9Vδ2 T cells in HD-PBMC after treatment with Venetoclax, 5-Azacytidine or the combination for 48 h. N=4 HD. C) Mean±SEM frequencies of Live (black), Apoptotic (light grey) or Dead (dark grey) Vγ9Vδ2 T cells in HD-PBMC 48 h after treatment with Venetoclax in the presence of ICT01 (right) or its isotype control (hIgG1S, left). N=4 HD. D) Mean±SEM frequency of Live Vγ9Vδ2 T cells in HD-PBMC 48 h after treatment with Venetoclax in the presence of ICT01 (black) or its isotype control (hIgG1S, light grey), normalized to no Venetoclax conditions. N=4 HD, each dot represents one healthy donor. *** p<0,005, 2-way ANOVA. E) Mean±SEM CD69 median mean fluorescence intensity (MFI) of Vγ9Vδ2 T cells in HD-PBMC treated for 48 h with ICT01 (black) or its isotype control (hIgG1S, light grey). N=5 HD, each dot represents one healthy donor.

FIG. 2: Activation of Vγ9Vδ2 T Cells with ICT01 Protects Them from Bcl-2 Family Member Inhibitors (Navitoclax, ABT-737 and MIK665) Induced Cell Death. A) Heatmap showing the mean frequency of live Vγ9Vδ2 T cells in HD-PBMC assessed by flow cytometry after treatment with increasing concentration of the indicated Bcl-2 family member inhibitor for 48 h. N=3 HD for ABT-737 and N=5 for Navitoclax and MIK665. B-D) Frequency of live Vγ9Vδ2 T cells (Mean±SEM) in HD-PBMC assessed by flow cytometry after 48 h of treatment with indicated concentration of ABT-737 (N=3 HD) (B), Navitoclax (N=5 HD) (C), or MIK665 (N=5 HD) (D), in the presence of 1 μg/mL of ICT01 (black) or its isotype control hIgG1S (light grey). Data were normalized to no ABT-737, Navitoclax or MIK665 treatment and each dot represents one healthy donor. * p<0.05, ** p<0.01, *** p<0.001, **** p<0.0001, 2 way ANOVA.

FIG. 3: ICT01-Mediated Activation Prior to Treatment Decreases Venetoclax Sensitivity of Vγ9Vδ2 T Cells. A) Schematic representation of the treatment sequence. B) Mean±SEM frequency of CD25+Vγ9Vδ2 T cells in HD-PBMC on day 3 of treatment with ICT01 (black) or its isotype control (hIgG1S, light grey) and Venetoclax. N=6 HD. C) Mean±SEM relative number of Live Vγ9Vδ2 T cells and D) Mean±SEM % of Live Vγ9Vδ2 T cells relative to no Venetoclax condition on day 3 of treatment with ICT01 (black) or its isotype control (hIgG1 S, light grey) and Venetoclax. N=5 HD. Each dot represents one healthy donor. *p<0,05, 2-way ANOVA.

FIG. 4: Vγ9Vδ2 T Cell Phenotypic Activation and Proliferation in HD-PBMC Stimulated with ICT01 is Mainly Unaffected by Treatment with Venetoclax and 5-Azacytidine: A) Schematic representation of the treatment sequence (left) and Mean±SEM frequency of CD25 expressing Vγ9Vδ2 T cells in HD-PBMC on day 4 of treatment with Venetoclax, 5-Azacytidine or the combination in presence of ICT01 (black) or its isotype control (hIgG1S, light grey) (right). N=6 HD, each dot represents one healthy donor. B) Schematic representation of the treatment sequence (left) and Mean±SEM frequency of CD25 expressing Vγ9Vδ2 T cells in HD-PBMC on day 3 of treatment with Venetoclax, 5-Azacytidine or the combination followed by treatment with ICT01 (black) or its isotype control (hIgG1S, light grey) (right). N=6 HD, each dot represents one healthy donor. C and D) Mean±SEM frequency of Cell Trace Violet (CTV) dim Vγ9Vδ2 T cells in HD-PBMC on day 4 of treatment with Venetoclax, 5-Azacytidine or the combination and ICT01 (black) or its isotype control (hIgG1S, light grey) in the absence (C) or presence of IL2 (D) N=6 HD, each dot represents one healthy donor, *p<0,05, RN one way ANOVA.

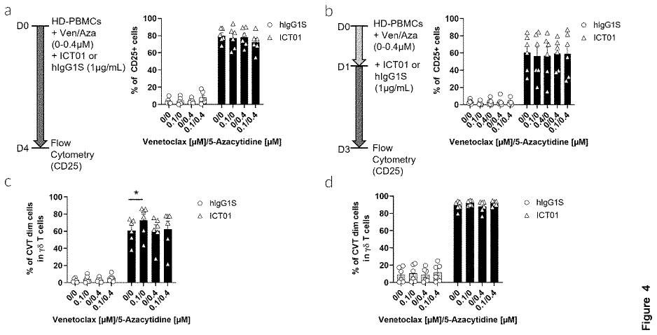

FIG. 5: Sequential Treatment with ICT01-Activated Vγ9Vδ2 T Cells Followed by Venetoclax and 5-Azacytidine Significantly Reduces Numbers of Resistant AML Cell Line Cells. A) Sensitivity of KG1a cell line to Venetoclax, 5-Azacytidine or the combination. Figure shows the Mean of luminescent signal relative to untreated conditions reflecting Live KG1a AML cells after 48 h of treatment with Venetoclax, 5-Azacytidine or the combination. N=2 experiments. B) Mean±SEM relative number of Live KG1a AML cells monitored by flow cytometry after 3 days of co-culture with HD-PBMC (E:T ratio 25:1) treated with ICT01 (black) or its isotype control (hIgG1S, light grey) at 0.1 or 1 μg/mL. N=6, each dot represents one healthy donor. C) Schematic representation of treatment schedule for killing of KG1a AML cells by HD-PBMC. D) Relative number of Live KG1a AML cells (Mean±SEM) (left) and Frequency of Live KG1a AML cell relative to no Venetoclax or 5-Azacytidine treatment (Mean±SEM) (right) after 3 days of co-culture with HD-PBMC in presence of ICT01 (black) or its isotype control (hIgG1S, light grey) and treatment with Venetoclax, 5-Azacytidine or the combination. N=6, each dot represents one healthy donor, * p<0.05, Wilcoxon test.

FIG. 6: Treatment with the Anti-BTN3A m20.1 and Venetoclax and 5-Azacytidine Significantly Improves Vγ9Vδ2 T cell-Mediated Killing of AML Cell Line KG1a. KG1a cells were co-cultured with HD-PBMC at ratio 5:1 in presence of m20.1 or isotype control (0.1 μg/mL). After 6 hrs, indicated concentrations of Venetoclax, 5-Azacytidine or the combination were added to the culture. Relative number of live KG1a was monitored after 48 hrs by flow cytometry. Figure shows the relative numbers of live KG1a AML cells (Mean±SEM) after 2 days of co-culture with HD-PBMC in presence of m20.1 (black) or its isotype control mIgG1 (light grey) and treatment with Venetoclax, 5-Azacytidine or the combination. N=6, each dot represents one healthy donor, * p<0.05, ** p<0.01, *** p<0.001, **** p<0.0001, 2 way ANOVA.

FIG. 7: Treatment with ICT01-Activated Vγ9Vδ2 T cells Followed by Venetoclax and 5-Azacytidine Significantly Reduces Numbers of Live Burkitt Lymphoma and Chronic-B-Cell Leukemia Cell Lines. Raji cells (Burkitt Lymphoma) or JVM2 cells (Chronic-B cell Leukemia) were co-cultured with HD-PBMC at ratio 5:1 in presence of ICT01 or isotype control (0.1 μg/mL or 1 μg/mL). After 6 hrs, indicated concentrations of Venetoclax, 5-Azacytidine or the combination were added to the culture. Relative numbers of live Raji (A) or JVM2 (B) were monitored after 48 hrs by flow cytometry. Figure shows the relative numbers (Mean±SEM) of live Raji (A) or JVM2 (B) after 2 days of co-culture with HD-PBMC in presence of ICT01 (black) or its isotype control hIgG1S (light grey) and indicated concentration of Venetoclax and 5-Azacytidine. N=6, each dot represents one healthy donor, * p<0.05, ** p<0.01, 2 way ANOVA.

FIG. 8: Treatment with ICT01-Activated Vγ9Vδ2 T Cells Followed by Inhibitors of Bcl-2 Family Members Significantly Reduces Numbers of Live AML Cell Lines. KG1a or MOLM14 AML cell lines were co-cultured with HD-PBMC at ratio 5:1 in presence of ICT01 or isotype control (0.1 μg/mL). After 6 hrs, indicated concentrations of ABT-737, Navitoclax or MIK665 were added to the culture. A) Relative numbers of live KG1a cells (Mean±SEM) after 2 days of co-culture with HD-PBMC in presence of ICT01 (black) or its isotype control hIgG1S (light grey) and treatment with indicated concentration of ABT-737. B) Relative numbers of live KG1a cells (Mean±SEM) after 2 days of co-culture with HD-PBMC in presence of ICT01 (black) or its isotype control hIgG1S (light grey) and treatment with indicated concentration of Navitoclax. C) Relative numbers of live MOLM14 cells (Mean±SEM) after 2 days of co-culture with HD-PBMC in presence of ICT01 (black) or its isotype control hIgG1S (light grey) and treatment with indicated concentration of MIK665. N=6 HD, each dot represents one healthy donor, * p<0.05, 2 way ANOVA.

FIG. 9: ICT01 combined with Venetoclax and 5-Azacytidine significantly prolongs survival of MOLM-14 engrafted NSG mice: Kaplan-Meier survival curves for each treatment group demonstrate the improved efficacy of combining ICT01 with Venetoclax and 5-Azacytidine. Statistical analysis were performed using the log-rank (Mantel-Cox) test. ** p<0.005, *** p<0.0005.

### DETAILED DESCRIPTION

### Definitions

In order that the present disclosure may be more readily understood, certain terms are first defined. Additional definitions are set forth throughout the detailed description.

The terms “polypeptide”, “protein” or “peptide” as used herein refer to any chain of amino acid residues, regardless of its length or post-translational modification (such as glycosylation).

As used herein, the term “BTN3A” has its general meaning in the art. In specific embodiments, it refers to human BTN3A polypeptides including either BTN3A1 of SEQ ID NO:24, BTN3A2 of SEQ ID NO:25 or BTN3A3 of SEQ ID NO:26.

The term “antibody” as used herein refers to immunoglobulin molecules and immunologically active portions of immunoglobulin molecules, i.e., molecules that contain an antigen binding site that immunospecifically binds an antigen. The term “antibody” or “immunoglobulin” have the same meaning and will be used equally in the present disclosure. As such, the term antibody encompasses not only whole antibody molecules, but also antibody fragments as well as variants (including derivatives) of antibodies. The term “antibody” as used herein also includes bispecific or multispecific molecules. An antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, an antibody of the disclosure can be functionally linked (e.g., by chemical coupling, genetic fusion, noncovalent association or otherwise) to one or more other binding molecules, such as another antibody, antibody fragment, peptide, or binding mimetic, such that a bispecific molecule results. Additionally, for the embodiment in which the bispecific molecule is multi-specific, the molecule can further include a third binding specificity, in addition to the first and second target epitope.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three-dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization. In some humanized forms of antibodies, some, most or all of the amino acids outside the CDR regions can be replaced with amino acids from human immunoglobulin molecules but where some, most or all amino acids within one or more CDR regions are unchanged. Small additions, deletions, insertions, substitutions, or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., Mol. Biol. 294:151, 1999.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

The term “antigen-binding fragment” of an antibody (or simply “antibody fragment”), as used herein, refers to full length or to one or more fragments of an antibody that retain the ability to specifically bind to an antigen (e.g., a BTN3A protein as above defined). In specific embodiments, an antibody provided herein is an antibody fragment, and more particularly any protein including an antigen-binding domain of an antibody as disclosed herein. Well known antibody fragments comprise: a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments; a diabody, which refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody (also shortly named herein antibody fragment). More generally antibody fragments as herein intended also encompass single-domain antibodies that are antibody fragments comprising all or a portion of the heavy chain variable domain or all or a portion of the light chain variable domain of an antibody. In certain embodiments, a single-domain antibody is a human single-domain antibody (Domantis, Inc., Waltham, M A; see, e.g., U.S. Pat. No. 6,248,516 B1). These antibody fragments are obtained using conventional techniques known to those of skill in the art, and the fragments are screened for utility in the same manner as are intact antibodies. Well-suited antibody fragments include, but are not limited to, Fv, Fab, F(ab′)2, Fab′, dsFv, scFv, sc(Fv)2 and diabodies. Antibody fragments can be made by various techniques, including but not limited to proteolytic digestion of an intact antibody as well as production by recombinant host cells as described herein.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

“Recombinant antibodies” are antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments a recombinant human antibody of this disclosure has the same amino acid sequence as the corresponding naturally occurring human antibody but differs structurally from said naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

An “isolated antibody”, as used herein, refers to an antibody that is substantially free of other antibodies having different antigenic specificities (e.g., an isolated antibody that specifically binds to BTN3A is substantially free of antibodies that specifically bind to other antigens than BTN3A). An isolated antibody that specifically binds to BTN3A may, however, have cross-reactivity to other antigens, such as related BTN3A molecules from other species. Moreover, an isolated antibody may be substantially free of other cellular material and/or chemicals.

The phrases “an antibody recognizing an antigen” and “an antibody having specificity for an antigen” are used interchangeably herein with the term “an antibody which binds specifically to an antigen”. The terms “an anti-BTN3A antibody” or “a BTN3A antibody” are also shortly used herein with the meaning of “an antibody recognizing BTN3A”.

As used herein, the term “activating antibody” refers to an antibody able to directly or indirectly induce immune functions of effector cells. In particular, as used herein, an activating anti-BTN3A antibody has at least the capacity to induce the activation of γδ T cells, typically Vγ9Vδ2 T cells, in co-culture with BTN3 expressing cells, with an EC50 below 5 μg/ml, preferably of 1 μg/ml or below, as measured in a degranulation assay (see for detailed assay WO/2020/025703).

As used herein, the term “binding” in the context of the binding of an antibody to a predetermined antigen or epitope, notably BTN3A, means typically a binding with an affinity corresponding to a K_{D} of about 10⁻⁷ M or less, such as about 10⁻⁸ M or less, such as about 10 ⁻⁹ M or less, about 10⁻¹⁰ M or less, or about 10⁻¹¹ M or even less when determined by for instance surface plasmon resonance (SPR) technology in a BIAcore 3000 instrument using typically a soluble form of the antigen as the ligand and the antibody as the analyte. BIACORE® (GE Healthcare, Piscataway, NJ) is one of a variety of surface plasmon resonance assay formats that are routinely used to epitope bin panels of monoclonal antibodies. Typically, an antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen.

The term “affinity”, as used herein in the context of an antibody, means the strength of the binding of an antibody to an epitope.

The term “Kₒₙ” or “Kass” (Kₐ), as used herein, is intended to refer to the association rate of a particular antibody-antigen interaction, whereas the term “K_{d} is” (Kd) or “K_{off},” as used herein, is intended to refer to the dissociation rate of a particular antibody-antigen interaction.

The term “K_{D}”, as used herein, is intended to refer to the equilibrium dissociation constant, which is obtained from the ratio of k_{off} to kₒₙ (i.e. k_{off}/kₒₙ) and is expressed as a molar concentration (M). The K_{D} value relates to the concentration of antibody (the amount of antibody needed for a particular experiment) and so the lower the K_{D} value (lower concentration) and thus the higher the affinity of the antibody. K_{D} values for antibodies can be determined using methods well established in the art. Preferred methods for determining the K_{D} values of mAbs can be found in Harlow, et al., Antibodies: A Laboratory Manual, Cold Spring Harbor Laboratory Press, Cold Spring Harbor, N.Y., (1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. A method for determining the K_{D} of an antibody is by using surface plasmon resonance, or by using a biosensor system such as a Biacore® (see also for detailed information regarding affinity assessment Rich R L, Day Y S, Morton T A, Myszka D G. High-resolution and high-throughput protocols for measuring drug/human serum albumin interactions using BIACORE®. Anal Biochem. 2001 Sep. 15; 296(2):197-207) or Octet® systems. The Octet® platform is based on bio-layer interferometry (BLI) technology. The principle of BLI technology is based on the optical interference pattern of white light reflected from two surfaces—a layer of immobilized protein and an internal reference layer. The binding between a ligand immobilized on the biosensor tip surface and an analyte in solution produces an increase in optical thickness at the biosensor tip, which results in a shift in the interference pattern measured in nanometers. The wavelength shift (AA) is a direct measure of the change in optical thickness of the biological layer, when this shift is measured over a period of time and its magnitude plotted as a function of time, a classic association/dissociation curve is obtained. This interaction is measured in real-time, allowing to monitor binding specificity, association rate and dissociation rate, and concentration (see Abdiche et al. 2008 but also the details in the results). Affinity measurements are typically performed at 25° C.

As used herein, the term “specificity” refers to the ability of an antibody to detectably bind an epitope presented on an antigen, such as a BTN3A. In some embodiments, it is intended to refer to an antibody that binds to human BTN3A as expressed on peripheral blood mononuclear cells (PBMCs), preferably with an EC₅₀ below 50 μg/ml and more preferably below 10 μg/ml as determined in the Examples (assays and protocols are typically disclosed in WO/2020/025703, in particular with reference to Table 4). In other embodiments, it binds to an antigen recombinant polypeptide with a K_{D} of 100 nM or less, 10 nM or less, 1 nM or less, 100 pM or less, or 10 pM or less, as measured by SPR measurements as mentioned above but see also for details Table 4 of WO/2020/025703).

An antibody that “cross-reacts with an antigen other than BTN3A” is intended to refer to an antibody that binds that antigen other than BTN3A with a K_{D} of 10 nM or less, 1 nM or less, or 100 pM or less. An antibody that “does not cross-react with a particular antigen” is intended to refer to an antibody that binds to that antigen, with a K_{D} of 1 μM or greater, or a K_{D} of 10 μM or greater. In certain embodiments, such antibodies that do not cross-react with the antigen exhibit essentially undetectable binding against these proteins in standard binding assays. In specific embodiments, the humanized antibody of the present disclosure, e.g., mAb1, cross-reacts with cynomolgus BTN3A1, BTN3A2 and BTN3A3 of SEQ ID NO:27, SEQ ID NO:28 and SEQ ID NO:29 respectively for example as measured in Biacore assay (see notably the related assay exemplified in WO/2020/025703 with reference to Table 26).

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN3A polypeptide).

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the term, “optimized” means that a nucleotide sequence has been altered to encode an amino acid sequence using codons that are preferred in the production cell or organism, generally a eukaryotic cell, for example, a Chinese Hamster Ovary cell (CHO) or a human cell. The optimized nucleotide sequence is engineered to retain completely or as much as possible the amino acid sequence originally encoded by the starting nucleotide sequence.

The amino acid sequences encoded by optimized nucleotide sequences are also referred to as optimized.

The term “identity” as used herein in reference to polypeptide sequences, refers to the amino acid sequence identity between two molecules. When an amino acid position in both molecules is occupied by the same amino acid, then the molecules are identical at that position. The identity between two polypeptides is a direct function of the number of identical positions. In general, the sequences are aligned so that the highest order match is obtained (including gaps if necessary). The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences can be determined using published techniques and widely available computer programs, such as BLASTP, FASTA (Atschul et al., J. Molecular Biol. 215:403, 1990), or the Needleman and Wunsch (J. Mol, Biol. 48:444-453, 1970) algorithm which has been incorporated into the GAP program in the GCG software package (Devereux et al., Nucleic Acids Res. 12:387, 1984, typically available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

Additional antibodies can be identified based on their ability to cross-compete (e.g., to competitively inhibit the binding of, in a statistically significant manner) with other antibodies of the disclosure in standard antigen binding assays such as an ELISA binding assay. The ability of a test antibody to inhibit the binding of antibodies of the present disclosure to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the disclosure provides antibodies that bind to the same antigen as, and compete with, the antibodies disclosed herein. As used herein, an antibody “competes” for binding when the competing antibody inhibits the target binding of an antibody or antigen binding fragment of the disclosure by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% in the presence of an equimolar concentration of competing antibody.

“combination therapy”, “co-administration”, “combined administration” or “concomitant administration” refers to a combined administration of at least two therapeutic agents, where a first agent, typically a BTN3A activating compound is administered at the same time or separately within time intervals, with a second agent, e.g. a Bcl2 family inhibitor and optionally a third agent, e.g. a hypomethylating agent, in the same subject in need thereof, where these time intervals allow that the combined partners show a cooperative or synergistic effect for treating a disorder, e.g. cancer and more specifically hematological malignancies. It is not intended to imply that the therapeutic agents must be administered at the same time and/or formulated for delivery together although these methods of delivery are within the scope described herein. The BTN3A activating antibody, e.g. ICT01 as herein disclosed, can be administered concurrently with or prior to, or subsequent to one or more other additional therapies or therapeutic agents. The terms are also meant to encompass treatment regimens in which the agents are not necessarily administered by the same route of administration.

As used herein the term synergy or synergistic effect when used in connection with a description of the efficacy of a combination of agents, means any measured effect of the combination which is greater than the effect predicted from a sum of the effects of the individual agents (i.e., greater than an additive effect). In some embodiments, the rate of tumor growth or tumor size (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In some embodiments, median overall survival time (e.g. of <12 months) is used to determine whether a combination of drugs is synergistic (e.g., a combination of drugs is synergistic when the median overall survival time of a subject or population of subjects is longer than would be expected if the combination of drugs produced an additive effect). In some embodiments, complete remission (CR) and complete remission with incomplete count recovery (CRi) rates is used to determine whether a combination of drugs is synergistic (as compared the monotherapy). In some embodiments, T cell expansion can also be used to determine whether a combination of drugs is synergistic (e.g., a combination of drugs is synergistic when the expansion rate (determined as increased percentage of the population or increased absolute cell number compared to a baseline value) of a specific T cell subset is higher than would be expected if the combination of drugs produced an additive effect).

### Activating BTN3A Antibody

An activating anti-BTN3A antibody according to the present disclosure typically exhibits one or more of the following properties:
(i) it binds to BTN3A with a K_{D} of 10 nM or less, preferably with a K_{D} of 1 nM or less, as measured by SPR, for example as described in the Examples of patent application WO2020025703;
(ii) it cross-reacts to cynomolgus BTN3A with a K_{D} of 100 nM or less, preferably with a K_{D} of 10 nM or less, as measured by SPR, for example as described in the Examples of patent application WO2020025703;
(iii) it binds to human PBMCs with an EC₅₀ of 50 μg/ml or below, preferably of 10 μg/ml or below, as measured in a flow cytometry assay as described in the Examples of patent application WO2020025703;
(iv) it induces the activation of γδ-T cells, typically Vγ9Vδ2 T cells, in co-culture with BTN3A expressing cells, with an EC₅₀ below 5 μg/ml, preferably of 1 μg/ml or below, as described in the Examples of patent application WO2020025703;
(v) it induces in vitro activation of Vγ9Vδ2 T cells in human PBMC, with an EC₅₀ below 0.1 μg/mL, preferably of 0.01 μg/mL or below, e.g., between 100 μg/mL and 0.1 μg/mL, as measured by surface expression of the activation markers CD69.

Examples of BTN3A activating antibodies are described in the paragraphs below. In some embodiments, the BTN3A activating antibody is selected from the group consisting of BTN3A antibody such as described in the International Patent Applications WO2012080769; WO2012080351, and WO2020025703. In some particular embodiments, BTN3A activating antibody is selected from the humanized antibodies described in WO2020025703 or is a humanized version of the BTN3A activating antibodies described in WO2012080769 and WO2012080351. In some embodiments, the BTN3A activating antibody can be selected from mAb 20.1, and mAb 7.2, which are obtainable from one of the hybridomas accessible under CNCM deposit number I-4401, and I-4402 such as described in WO2012080769 and WO2012080351 or humanized version thereof, as well as from humanized mAbs 1-6 described in WO2020025703.

In some embodiments, the BTN3A activating antibody comprises the six CDRs (CDR1 (also called HCDR1), VH CDR2 (also called HCDR2), VH CDR3 (also called HCDR1), VL CDR1 (also called LCDR1), VL CDR2s (also called LCDR2), VL CDR3s (also called HCDR3)) of the antibodies 20.1, or 7.2 described in WO2012080769 and WO2012080351 or mAbs 1-6 as described in WO2020025703. In particular embodiments, the anti-BTN3A activating antibody comprises HCDR1, HCDR2, HCDR3, LCDR1, LCDR2, and HCDR3 as shown in Table 1 below:

TABLE 1
CDR regions of mAb1, mAb2, mAb4 and mAb5, parental
murine mAb 7.2 and murine mAb 20.1 antibody according
to Kabat numbering as defined in WO2020025703.

| Original | | | | | | |
|---|---|---|---|---|---|---|
| antibody | HCDR1 | HCDR2 | HCDR3 | LCDR1 | LCDR2 | LCDR3 |
| mAb 7.2 | SEQ ID | SEQ ID | SEQ ID | SEQ ID | SEQ ID | SEQ ID |
| mAb1 (ICT01) | NO: 12 | NO: 13 | NO: 14 | NO: 15 | NO: 16 | NO: 17 |
| mAb2 | | | | | | |
| mAb4 | | | | | | |
| mAb5 | | | | | | |
| mAb 20.1 | SEQ ID | SEQ ID | SEQ ID | SEQ ID | SEQ ID | SEQ ID |
| | NO: 18 | NO: 19 | NO: 20 | NO: 21 | NO: 22 | NO: 23 |

In some embodiments of antibodies for use as herein disclosed, the 6 CDR regions are 100% identical to the 6 CDR regions of the antibodies 20.1, or 7.2 described in WO2012080769 and WO2012080351 or of mAbs 1-6 described in WO2020025703, notably in some embodiments, the 6 CDR regions of antibodies as herein disclosed are 100% identical to the 6 CDR regions of Table 1, notably of mAbs7.2;1; 2; 4; and 5.

Other antibodies for use as disclosed herein include those having amino acids that have been mutated by amino acid deletion, insertion or substitution, yet have at least 60, 70, 80, 90, 95, 96, 97, 98, 99 or 100 percent identity in the CDR regions as compared to the 6 CDR regions of the antibodies 20.1, or 7.2 described in WO2012080769 and WO2012080351 or of mAbs 1-6 described in WO2020025703, notably as compared to the 6 CDR regions defined in Table 1. Typically, as per the present disclosure, antibodies may have between 1, 2, 3 or 4 amino acid variations (including deletion, insertion, or substitution) in one or more CDRs, as compared to the CDR sequences of the antibodies 20.1, or 7.2 described in WO2012080769 and WO2012080351 or of mAbs 1-6 described in WO2020025703, notably as compared to the CDR sequences of Table 1, more particularly as compared to the CDR sequences of mAbs 7.2; 1; 2; 4; and 5.

In specific embodiments, the antibodies for use according to the present disclosure comprises a variant CDRH2 of mAb 20.1 having the substitution N5S and K10N (also referred as N53S, K58N according to Kabat numbering).

In other specific embodiments, the antibodies for use according to the present disclosure comprises a variant CDRL1 of mAb 20.1 having the substitution of L8V (also referred as L31V according to Kabat numbering).

In more specific embodiments, the antibodies for use according to the present disclosure comprises a variant CDRH2 of mAb 20.1 having the substitution N5S and K10N (also referred as N53S, K58N according to Kabat numbering) and a variant CDRL1 of mAb20.1 having the substitution of L8V (also referred as L31V according to Kabat numbering).

In a specific embodiment, BTN3A activating antibodies for use according to the present disclosure comprises heavy chain CDR1-3 sequences of SEQ ID NO:34, SEQ ID NO:35 and SEQ ID NO:36 respectively, and light chain CDR1-3 sequences of of SEQ ID NO:37, SEQ ID NO:38 and SEQ ID NO:39.

In a specific embodiment, BTN3A activating antibodies for use according to the present disclosure comprises a variable heavy chain VH of SEQ ID NO:40, and a variable light chain of SEQ ID NO:41.

In specific embodiments, said BTN3A activating antibody is a bispecific antibody characterized in comprising a first binding part specifically binding to human BTN3A and a second binding part specifically binding to a tumor antigen, typically a tumor antigen targeting a malignant cell of said targeted hematological malignancy. For example, said first binding part is a full-length bivalent antibody having the heavy chain CDR sequences CDRH1 of SEQ ID NO:34, CDRH2 of SEQ ID NO:35, CDRH3 of SEQ ID NO:36, and the light chain CDR sequences CDRL1 of SEQ ID NO:37, CDRL2 of SEQ ID NO:38, CDRL3 of SEQ ID NO:39.

Other anti-BTN3A activating antibodies, in particular variant of mAb20.1 activating antibodies are disclosed in WO2023/161457 (Evobright GmbH) which content is incorporated herein in its entirety. Antibodies for use of the present disclosure include also those having at least 90%, notably at least, 95, 96, 97, 98, 99 or 100% identity with the VH and VL regions as defined in Table 2. More particularly, antibodies of the disclosure include the selected humanized recombinant antibodies mAb1, mAb2, mAb4 and mAb5, which are structurally characterized by their variable heavy and light chain amino acid sequences and human constant regions (isotypes) as described in the Table 2 below:

TABLE 2
Variable heavy and light chain amino
acid sequences of mAb1-mAb6

| | VH | VL | |
|---|---|---|---|
| | Amino acid | Amino acid | |
| Antibody | sequence | sequence | Isotype constant region |
| mAb1 | SEQ ID NO: 1 | SEQ ID NO: 2 | Silent IgG1 |
| (ICT01) | (VH2 7.2) | (Vk1 7.2) | L247F/L248E/P350S |
| mAb2 | SEQ ID NO: 1 | SEQ ID NO: 3 | Silent IgG1 |
| | (VH2 7.2) | (Vk2 7.2) | L247F/L248E/P350S |
| mAb3 | SEQ ID NO: 30 | SEQ ID NO: 31 | Silent IgG1 |
| | | | L247F/L248E/P350S |
| mAb4 | SEQ ID NO: 1 | SEQ ID NO: 2 | IgG4 S241P/L248E |
| | (VH2 7.2) | (Vk1 7.2) | |
| mAb5 | SEQ ID NO: 1 | SEQ ID NO: 3 | IgG4 S241P/L248E |
| | (VH2 7.2) | (Vk2 7.2) | |
| mAb6 | Same as mAb3 | Same as mAb3 | IgG4 S241P/L248E |

mAb3 and mAb6 are humanized antibodies of parental murine BTN3A activating antibody, referred to as mAb 20.1 described in WO2012/080351.

The corresponding amino acid and nucleotide coding sequence of the constant isotype regions of IgG1, IgG4 and their mutant versions IgG1 L247F/L248E/P350S and IgG4 S241P/L248E used for generating mAb1 to mAb6 are well-known in the art (Oganesyan et al., 2008; Reddy et al., 2000). The C-terminal lysine found in IgG may be naturally cleaved off and this modification does not affect the properties of the antibody; so, this residue may additionally be deleted in the constructs of mAb1 to mAb6.

Full length light and heavy chains and corresponding coding sequences of mAb1, mAb2, mAb4 and mAb 5 are shown in the Table 3 below.

TABLE 3
Full length heavy and light chain DNA coding sequences

| Antibody | Amino acid sequence | DNA coding sequence |
|---|---|---|
| mAb1 | Heavy Chain: SEQ ID NO: 4 | Heavy Chain: SEQ ID NO: 8 |
| (ICT01) | Light Chain: SEQ ID NO: 6 | Light Chain: SEQ ID NO: 10 |
| mAb2 | Heavy Chain: SEQ ID NO: 4 | Heavy Chain: SEQ ID NO: 8 |
| | Light Chain: SEQ ID NO: 7 | Light Chain: SEQ ID NO: 11 |
| mAb3 | Heavy Chain: SEQ ID NO: 32 | — |
| | Light Chain: SEQ ID NO: 33 | |
| mAb4 | Heavy Chain: SEQ ID NO: 5 | Heavy Chain: SEQ ID NO: 9 |
| | Light Chain: SEQ ID NO: 6 | Light Chain: SEQ ID NO: 10 |
| mAb5 | Heavy Chain: SEQ ID NO: 5 | Heavy Chain: SEQ ID NO: 9 |
| | Light Chain: SEQ ID NO: 7 | Light Chain: SEQ ID NO: 11 |

In certain embodiments that may be combined with the previous embodiments, an antibody provided herein is an antibody fragment of the above-defined antibodies. Antibody fragments include for example, but are not limited to, Fab, Fab′, Fab′-SH, F(ab′)2, Fv, Unibody, and scFv fragments, diabodies, single domain or nanobodies and other fragments. Preferably, it is a monovalent antibody, such as a Fab of scFv fragments.

In some embodiments, the antibodies of the present disclosure compete for binding to BTN3A antibodies described above, in particular an antibody of the present disclosure competes for binding with an antibody selected from mAb 20.1, and mAb 7.2, which are obtainable from one of the hybridomas accessible under CNCM deposit number I-4401, and I-4402 such as described in WO2012080769 and WO2012080351, as well as from mAbs 1-6 described in WO2020025703. In more particular embodiments, the antibodies of the present disclosure compete for binding with an antibody selected from mAb 7.2 as produced by the hybridomas deposited at the CNCM under deposit number I-4402, and an antibody having a heavy chain of SEQ ID NO:4 and a light chain of SEQ ID NO:6.

In some embodiments, antibodies of the present disclosure are chimeric, humanized, or human antibodies. In preferred embodiments of the present disclosure, the BTN3A antibody is a humanized antibody. Typically, a non-human antibody is humanized to reduce immunogenicity to humans, while having at least the same affinity (or superior affinity) of the parental non-human antibody. More particularly the BTN3A antibody is a humanized form of the antibodies 20.1, or 7.2 disclosed in WO2012080351. In preferred embodiments, the antibodies of the present disclosure are humanized antibodies of the parent antibody mAb 7.2 as disclosed in WO2012080351. Generally, a humanized antibody comprises one or more variable domains in which, CDRs, (or portions thereof) are derived from a non-human antibody, e.g., the murine mAbs 7.2, and FRs (or portions thereof) are derived from the murine antibody sequences with mutations to reduce immunogenicity. A humanized antibody optionally will also comprise at least a portion of a human constant region. Preferably, the recombinant antibody according to the disclosure is a humanized silent antibody, typically a humanized silent IgG1 or IgG4 antibody. Well-suited humanized anti-BTN3A antibodies according to the present disclosure are typically described in WO2020025703 and include mAbs having VH/VL polypeptides sequences of Table 2 and mAbs having light/heavy chains of Table 3.

As used herein, the term “silent” antibody refers to an antibody that exhibits no or low FcγR binding and/or C1q binding as measured in binding assays such as those described in WO2020025703. In one embodiment, the term “no or low FcγR and/or C1q binding” means that the silent antibody exhibits an FcγR and/or C1q binding that is at least below 50%, for example below 80% of the FcγR and/or C1q binding that is observed with the corresponding antibody with wild type human IgG1 or IgG4 isotype.

Framework or Fc engineering

The antibodies of the disclosure can include modifications made to framework residues within VH and VL, to decrease its immunogenicity.

In some specific embodiments, the antibody of the disclosure is a humanized monoclonal antibody of the parent murine antibody mAb 7.2, including at least the following amino acid mutations in the VH framework regions: V5Q; V11L; K12V; R66K; S74F; 175S; E81Q; S82AR; R82BS; R83T; D85E; T87S; L108S; and at least the following amino acid mutations in the Vκ framework regions: T5N; V15L; R18T; V191; K42N; A431; D70G; F73L; Q100G.

In other specific embodiments, the antibody of the disclosure is a humanized monoclonal antibody of the parent murine antibody mAb 7.2, including at least the following amino acid mutations in the VH framework regions as compared to mAb 7.2: V5Q; V11L; K12V; R66K; S74F; 175S; E81Q; S82AR; R82BS; R83T; D85E; T87S; L108S; and at least the following amino acid mutations in the Vκ framework regions: T5N; V15L; R18T; V191; K42N; A431; S63T; D70G; F73L; Q100G.

In addition to modifications made within the framework regions, the antibodies of the disclosure may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody of the disclosure may be chemically modified (e.g., one or more chemical moieties can be attached to the antibody) or be modified to alter its glycosylation, again to alter one or more functional properties of the antibody. Each of these embodiments is described in further detail below.

As used herein, the term “isotype constant region” or “Fc region” is used interchangeably to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody wherein the numbering is according to the EU numbering system. The C-terminal lysine (residue K447) of the Fc region may be removed, for example, during production or purification of the antibody or its corresponding codon deleted in the recombinant constructs. Accordingly, a composition of antibodies of the disclosure may comprise antibody populations with all K447 residues removed, antibody populations with no K447 residues removed, and antibody populations having a mixture of antibodies with and without the K447 residue.

In some specific embodiments, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In other embodiments, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In other embodiments, the Fc region is modified to decrease the ability of the antibody to mediate antibody dependent cellular cytotoxicity (ADCC) and/or to decrease the affinity of the antibody for an Fcγ receptor by modifying one or more amino acids. Such antibodies with decreased effector functions, and in particular decreased ADCC include silent antibodies.

In certain embodiments, the Fc domain of the IgG1 isotype is used. In some specific embodiments, a mutant variant of the IgG1 Fc fragment is used, e.g. a silent IgG1 Fc which reduces or eliminates the ability of the fusion polypeptide to mediate antibody dependent cellular cytotoxicity (ADCC) and/or to bind to an Fcγ receptor.

In certain embodiments, the Fc domain of the IgG4 isotype is used. In some specific embodiments, a mutant variant of the IgG4 Fc fragment is used, e.g. a silent IgG4 Fc which reduces or eliminates the ability of the fusion polypeptide to mediate antibody dependent cellular cytotoxicity (ADCC) and/or to bind to an Fcγ receptor.

Silenced effector functions can be obtained by mutation in the Fc constant part of the antibodies and have been described in the Art (Baudino et al., J. Immunol. 2008; Strohl, CO Biotechnology 20 2009). Examples of silent IgG1 antibodies comprise the triple mutant variant IgG1 L247F L248E P350S. Examples of silent IgG4 antibodies comprise the double mutant variant IgG4 S241P L248E.

In certain embodiments, the Fc domain is a silent Fc mutant preventing glycosylation at position 314 of the Fc domain. For example, the Fc domain contains an amino acid substitution of asparagine at position 314. An example of such amino acid substitution is the replacement of N314 by a glycine or an alanine.

In still other embodiments, the glycosylation of an antibody is modified. For example, an aglycoslated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Another modification of the antibodies herein that is contemplated by the present disclosure is pegylation or hesylation or related technologies. An antibody can be pegylated to, for example, increase the biological (e.g., serum) half-life of the antibody. To pegylate an antibody, the antibody, or fragment thereof, typically is reacting with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See for example, EP 0 154 316 by Nishimura et al. and EP 0 401 384 by Ishikawa et al.

Another possibility is a fusion of at least the antigen-binding region of the antibody of the disclosure to proteins capable of binding to serum proteins, such human serum albumin to increase half-life of the resulting molecule. Such approach is for example described in Nygren et al., EP 0 486 525.

In certain embodiments, the C-terminal lysine commonly present on human IgG heavy chain constant domains, is engineered out to reduce heterogeneity due to the cleavage of this reduce commonly observed during manufacturing or storage. Such modifications do not perceptible change the desirable functions of these antibodies, while conferring the benefit of stability to these molecules.

### Nucleic Acid Molecules Encoding Antibodies of the Disclosure

Also disclosed herein are the nucleic acid molecules that encode the anti-BTN3A antibodies of the present disclosure. Examples of variable light chain and heavy chain nucleotide sequences are those encoding the variable light chain and heavy chain amino acid sequences of any one of mAb1, mAb2, mAb4 and mAb5, the latter sequences being easily derived from the Table 1 and Table 2, and using the genetic code and, optionally taking into account the codon bias depending on the host cell species.

The present disclosure also pertains to nucleic acid molecules that derive from the latter sequences having been optimized for protein expression in mammalian cells, for example, CHO cell lines.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (Ausubel et al., 1988, Current Protocols in Molecular Biology (John Wiley & Sons)). A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding, for example, VH and VL segments are obtained, these DNA fragments can be further manipulated by standard recombinant DNA techniques, for example to convert the variable region genes to full-length antibody chain genes, to Fab fragment genes or to an scFv gene. In these manipulations, a VL- or VH-encoding DNA fragment (for example VL and VH as defined in Table 1) is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (Kabat et al., K. S. (1992). Sequences of Proteins of Immunological Interest (DIANE Publishing)) and DNA fragments encompassing these regions can be obtained by standard PCR amplification. The heavy chain constant region can be an IgG1, IgG2, IgG3, IgG4, IgA, IgE, IgM or IgD constant region. In some embodiments, the heavy chain constant region is selected among IgG1 isotypes, for example human IgG1 isotype. In other embodiments, the heavy chain constant region is selected among IgG4 isotypes, for example human IgG4 isotype. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as to a Fab light chain gene) by operatively linking the VL-encoding DNA to another DNA molecule encoding the light chain constant region, CL. The sequences of human light chain constant region genes are known in the art (Kabat et al., 1992, see supra) and DNA fragments encompassing these regions can be obtained by standard PCR amplification. The light chain constant region can be a kappa or a lambda constant region.

To create an scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (Bird et al., 1988 see supra; Huston et al., 1988, see supra; McCafferty, J., et al., 1990. Nature 348, 552-554).

### Generation of Transfectomas Producing Monoclonal Antibodies

Antibodies of the present disclosure can be produced in a host cell transfectoma using, for example, a combination of recombinant DNA techniques and gene transfection methods as is well known in the art (Morrison, 1985; Science 229, 1202-1207).

For example, to express the antibodies, or antibody fragments thereof, DNAs encoding partial or full-length light and heavy chains can be obtained by standard molecular biology or biochemistry techniques (e.g., DNA chemical synthesis, PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors disclosed herein carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel's publication (Goeddel, D. V. (1990). [1] Systems for heterologous gene expression. In Methods in Enzymology, (Academic Press), pp. 3-7). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988, Mol. Cell. Biol. 8, 466-472).

In addition to the antibody chain genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the present disclosure in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

In one specific embodiment, a cloning or expression vector according to the disclosure comprises one of the coding sequences of the heavy and light chains of any one of mAb1, mAb2, mAb4 and mAb5 operatively linked to suitable promoter sequences.

Mammalian host cells for expressing the recombinant antibodies of the disclosure include Chinese Hamster Ovary (CHO) cells including dhfr− CHO cells (described in Urlaub and Chasin, 1980) used with a DHFR selectable marker (as described in Kaufman and Sharp, 1982), CHOK1 dhfr+ cell lines, NSO myeloma cells, COS cells and SP2 cells, for example GS CHO cell lines together with GS Xceed™ gene expression system (Lonza). When recombinant expression vectors encoding antibody genes are introduced into mammalian host cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007, J. Chromatogr. B 848, 28-39).

In one specific embodiment, the host cell of the disclosure is a host cell transfected with an expression vector having the coding sequences suitable for the expression of mAb1, mAb2, mAb4 and mAb5 respectively, operatively linked to suitable promoter sequences.

For example, the present disclosure relates to a host cell comprising at least the nucleic acids of SEQ ID NO:8 and 10 encoding respectively the heavy and light chains of mAb1.

The latter host cells may then be further cultured under suitable conditions for the expression and production of an antibody of the disclosure selected from the group consisting of mAb1, mAb2, mAb3, mAb4 and mAb5 respectively.

Alternatively, cell free expression systems may be used for the production of any of mAb1, mAb2, mAb3, mAb4 and mAb5. Typically, methods of cell-free expression of proteins or antibodies are already described (Stech et al., 2017, Sci. Rep. 7, 12030).

### Anti-BTN3A Immunoconjugates

In another aspect, the present disclosure features an anti-BTN3A antibody as disclosed herein, or a fragment thereof, conjugated to a therapeutic moiety. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells.

Cytotoxins can be conjugated to antibodies of the present disclosure using linker technology available in the art. Examples of linker types that have been used to conjugate a cytotoxin to an antibody include, but are not limited to, hydrazones, thioethers, esters, disulfides, and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Panowski et al., 2013 for a review on antibody drug conjugates.

Antibodies of the present disclosure can also be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰, and lutetium¹⁷⁷. Method for preparing radioimmunoconjugates are established in the art.

### Bispecific or Multispecific Anti-BTN3A Antibodies

In another aspect, it is further disclosed herein bispecific or multispecific molecules comprising an anti-BTN3A antibody of the present disclosure. An antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, an antibody of the disclosure can be functionally linked (e.g., by chemical coupling, genetic fusion, noncovalent association or otherwise) to one or more other binding molecules, such as another antibody, antibody fragment, peptide, or binding mimetic, such that a bispecific molecule results.

Accordingly, the present disclosure includes bispecific molecules comprising at least one first binding specificity for BTN3A, for example, one antigen-binding portion of any one of mAb1, mAb2, mAb3, mAb4, mAb5 and mAb6 (or functional variants thereof) and a second binding specificity for a second target epitope.

Additionally, for the embodiment in which the bispecific molecule is multi-specific, the molecule can further include a third binding specificity, in addition to the first and second target epitope.

In one embodiment, the bispecific molecules as disclosed herein comprise as a binding specificity at least one antibody, or an antibody fragment thereof, including, e.g., a Fab, Fab′, F(ab′)₂, Fv, Unibody or a single chain Fv. The antibody may also be a light chain or heavy chain dimer, or any minimal fragment thereof such as a Fv or a single chain construct as described in Ladner et al. U.S. Pat. No. 4,946,778.

Other antibodies which can be employed in the bispecific molecules disclosed herein are murine, chimeric, and humanized monoclonal antibodies.

The bispecific molecules of the present disclosure can be prepared by conjugating the constituent binding specificities, using methods known in the art. For example, each binding-specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (Karpovsky et al., 1984 J. Exp. Med. 160, 1686-1701; Liu et al., 1985 Proc. Natl. Acad. Sci. 82, 8648-8652). Other methods include those described in Brennan et al., 1985, Science 229, 81-83; Glennie, et al. 1987. J. Immunol. 139, 2367-2375; and Paulus, 1985 Behring Inst. Mitt. 118-132.

Alternatively, both binding specificities can be encoded in the same vector and expressed and assembled in the same host cell. This method is particularly useful where the bispecific molecule is a mAb×mAb, mAb×scFv, mAb×Fab, Fab×F(ab′)₂ or ligand ×Fab fusion protein.

In specific embodiments, a bispecific molecule can include a fusion of an anti-BTN3A antibody including full length heavy and light chains, and scFv binding to target epitope. In a related more specific embodiment, the scFv is fused at the C-terminal parts of the heavy and light chains of the antibody (bivalent binding specificities to the target epitope). A bispecific molecule of the disclosure can be a single chain molecule comprising one single chain antibody and a binding determinant, or a single chain bispecific molecule comprising two binding determinants.

Binding of the bispecific molecules to their specific targets can be confirmed by, for example, enzyme-linked immunosorbent assay (ELISA), radioimmunoassay (REA), FACS analysis, bioassay (e.g., growth inhibition and apoptosis), or Western Blot assay. Each of these assays generally detects the presence of protein-antibody complexes of particular interest by employing a labeled reagent (e.g., an antibody) specific for the complex of interest.

### Bcl2 Family Inhibitor

The present disclosure relates to the use of an activating BTN3A antibody in combination with at least one Bcl2 family inhibitor.

Apoptosis is induced in cells by the caspase activation cascade which can be triggered through extrinsic and intrinsic pathways. The intrinsic pathway is led by the disruption of mitochondrial integrity that is regulated by the BCL-2 family proteins. B-cell lymphoma 2 (BCL-2) was the first protein to be identified of this family, which gave it its name. Members are characterized by the expression of four BCL-homology (BH) domains. Six anti-apoptotic (BCL-2, BCL-XL, BCL-w, BCL-2-related protein A1 (Bfl-1/A1), myeloid cell leukemia 1 (MCL-1), and BCL-B/Boo) and ten pro-apoptotic family members (among them BAX and BAK) have been identified in humans. At homeostatic conditions, anti-apoptotic proteins bind the key apoptosis inducing proteins BAX and BAK, which ensures cell survival. Upon stress, such as loss of survival signals, DNA damage, chemicals, or therapeutic agents this balance is disturbed, mitochondrial membrane integrity lost, and apoptosis initiated (Perini et al., 2018 J Hematol Oncol. 11(1):65; Roberts, Hematology Am Soc Hematol Educ Program. 2020 Dec. 4; 2020(1):1-9).

As used herein, the term Bcl2 family inhibitor refers to (i) a BCL-2 and BCL-XL inhibitors; (ii) a selective BCL-2 inhibitor and (iii) an inhibitor of MCL-1.

In an embodiment, the Bcl2 family inhibitor is a Bcl2 inhibitor chosen among (i) a BCL-2 and BCL-XL inhibitor; and (ii) a selective BCL-2 inhibitor.

In specific embodiments, inhibitors of the BCL-2 family protein include inhibitors of MCL-1 such as AZD5991 (e.g., CAS number 2143010-83-5) and S64315 (MIK665) (e.g., CAS number 1799631-75-6).

In specific embodiments, a Bcl2 inhibitor is selected from BCL-2 and BCL-XL inhibitors include: ABT-737 (e.g., CAS number 852808-04-9), Navitoclax (e.g., CAS number 923564-51-6) and AZD4320 (e.g., CAS number 1357576-48-7).

Preferably, Bcl-2 inhibitors are selected from the group consisting of Venetoclax, Navitoclax, Obatoclax, even more preferably Venetoclax.

Venetoclax (DCI), also called ABT-199, selectively binds and inhibits the B-cell lymphoma-2 (BCL-2) protein. In some blood cancers, BCL-2 prevents cancer cells from undergoing apoptosis. IUPAC name of Venetoclax is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide.

Venetoclax has the following formula:

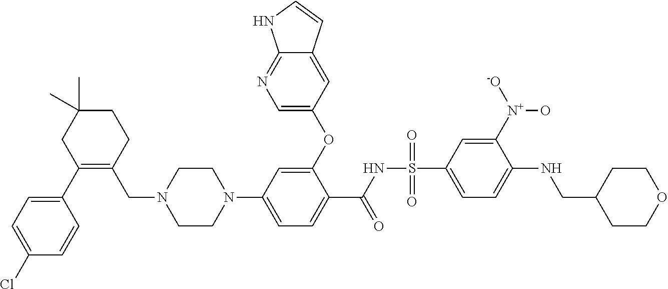

Venetoclax is marketed as VENCLEXTA™, which is in the form of a tablet. Venetoclax is indicated in the US: (i) for the treatment of adult patients with chronic lymphocytic leukemia (CLL) or small lymphocytic lymphoma (SLL); (ii) in combination with injectable 5-Azacytidine or Decitabine or low-dose Cytarabine for the treatment of newly diagnosed acute myeloid leukemia (AML) in adults who are age 75 years or older, or who have comorbidities that preclude use of intensive induction chemotherapy. In some embodiments, patients receive 20 mg/m² of Cytarabine once a day subcutaneously for 10 consecutive days every 4 weeks as the low-dose Cytarabine.

Navitoclax, also called ABT-263, selectively binds to apoptosis suppressor proteins Bcl-2, Bcl-XL, and Bcl-w and prevents their binding to the apoptotic effectors Bax and Bak proteins. IUPAC name of Navoticlax is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide.

Obatoclax, also called GX15-070, is a pan-inhibitor of Bcl-2 family proteins, with pro-apoptotic activity. It is a selective antagonist of the BH3-binding groove of the Bcl-2 family proteins, which are overexpressed in some cancers. IUPAC name of Obatoclax is (2Z)-2-[(5Z)-5-[(3,5-dimethyl-1H-pyrrol-2-yl)methylidene]-4-methoxypyrrol-2-ylidene]indole.

S64315, also called MIK665, is a highly potent and selective inhibitor of MCL-1 with pro-apoptotic and antineoplastic activities due to high expression of MCL-1 in a variety of human cancers including those of hematopoietic and lymphoid origin. IUPAC name of S64315 is (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoic acid.

In preferred embodiments, said Bcl-2 inhibitor for use in the combination of the present disclosure is Venetoclax.

### Hypomethylating Agents

In certain embodiments, the combined therapy described herein includes a hypomethylating agent. Hypomethylating agents are also known as HMAs or demethylating agents, which inhibits DNA methylation. In certain embodiments, the hypomethylating agent blocks the activity of DNA methyltransferase. In certain embodiments, the hypomethylating agent comprising but not restricted to Azacytidine, Decitabine.

Azacytidine is also known as 5-AC, 5-azacytidine, azacitidine, ladakamycin, 5-AZC, AZA-CR, U-18496, 4-amino-1-beta-D-ribofuranosyl-1,3,5-triazin-2(1H)-one, 4-amino-1-[(2R,3R,4S,5R)3,4-dihydroxy-5-(hydroxymethyl)oxolan-2-yl]-1,3,5-triazin-2-one, or VIDAZA®.

Azacytidine is a pyrimidine nucleoside analogue of cytidine with antineoplastic activity. Azacytidine is incorporated into DNA, where it reversibly inhibits DNA methyltransferase, thereby blocking DNA methylation. Hypomethylation of DNA by Azacytidine can activate tumor suppressor genes silenced by hypermethylation, resulting in an antitumor effect. Azacytidine can also be incorporated into RNA, thereby be disrupting normal RNA function and impairing tRNA cytosine-5-methyltransferase activity.

In some embodiments, Azacytidine is administered at a dose of about 25 mg/m² to about 150 mg/m², e.g., about 50 mg/m² to about 100 mg/m², about 70 mg/m² to about 80 mg/m², about 50 mg/m² to about 75 mg/m², about 75 mg/m² to about 125 mg/m², about 50 mg/m², about 75 mg/m², about 100 mg/m², about 125 mg/m², or about 150 mg/m². In some embodiments, Azacytidine is administered once a day. In some embodiments, Azacytidine is administered intravenously. In other embodiments, Azacytidine is administered subcutaneously. In some embodiments, Azacytidine is administered at a dose of about 50 mg/m² to about 100 mg/m² (e.g., about 75 mg/m²), e.g., for about 5-7 consecutive days, e.g., in a 28-day cycle. For example, Azacytidine can be administered at a dose of about 75 mg/m² for seven consecutive days on days 1-7 of a 28-day cycle. As another example, Azacytidine can be administered at a dose of about 75 mg/m² for five consecutive days on days 1-5 of a 28-day cycle, followed by a two-day break, then two consecutive days on days 8-9. As yet another example, Azacytidine can be administered at a dose of about 75 mg/m² for six consecutive days on days 1-6 of a 28-day cycle, followed by a one-day break, then one administration on day 8 will be permitted.

### Combination Kits and Compositions

### Combination Kits

The combination of the present disclosure comprising an anti-BTN3A antibody and a Bcl2 family inhibitor, for example a Bcl2 inhibitor, as previously defined may be presented as a combination kit. By the term “combination kit” or “kit of parts” as used herein is meant the pharmaceutical composition or compositions that are used to administer, the BTN3A activating antibody (e.g., mAb1) and the Bcl2 family inhibitor for example a Bcl2 inhibitor (e.g., Venetoclax), according to the disclosure. When both compounds are administered simultaneously, the combination kit can contain, for example, the components, suitably the anti-BTN3A antibody and the Bcl2 family inhibitor in separate pharmaceutical compositions. When the components, suitably the BTN3A activating antibody and the Bcl2 inhibitor are not administered simultaneously, the combination kit will contain the active principles in separate pharmaceutical compositions either in a single package or in separate pharmaceutical compositions in separate packages.

In one aspect there is provided a kit of parts comprising:
(i) the BTN3A activating antibody, e.g. mAb1, in association with a pharmaceutically acceptable excipients, diluents or carrier, typically a composition comprising the BTN3A activating antibody as previously defined; and
(ii) the Bcl2 inhibitor, e.g. Venetoclax, in association with a pharmaceutically acceptable excipients, diluents and/or carriers, typically a composition comprising the Bcl2 inhibitor as previously defined; and
(iii) optionally, a hypomethylating agent, such as Azacytidine or Decitabine.

In one embodiment of the present disclosure the kit of parts comprises:
(i) the BTN3A activating antibody, e.g. mAb1, in association with a pharmaceutically acceptable excipients, diluents or carrier, typically a composition comprising the BTN3A activating antibody as previously defined; and
(ii) the Bcl2 inhibitor, e.g. Venetoclax, in association with a pharmaceutically acceptable excipients, diluents and/or carriers, typically a composition comprising the Bcl2 inhibitor as previously defined; and
(iii) optionally, a hypomethylating agent, such as Azacytidine or Decitabine, in association with a pharmaceutically acceptable excipients, diluents and/or carriers,
   wherein the components are provided in a form which is suitable for sequential, separate and/or simultaneous administration.

In one embodiment the kit of parts comprises:
(i) a first container comprising the BTN3A activating antibody in association with pharmaceutically acceptable excipients, diluents and/or carrier, typically a composition comprising the BTN3A activating antibody as previously defined; and
(ii) a second container comprising the Bcl2 family inhibitor, for example a Bcl2 inhibitor, in association with pharmaceutically acceptable excipients, diluents and/or carriers, typically a composition comprising the Bcl2 family inhibitor, for example a Bcl2 inhibitor, as previously defined; and
(iii) optionally, a third container comprising a hypomethylating agent, such as Azacytidine or Decitabine, as previously defined.

The combination kit can also be provided by instruction, such as dosage and administration instructions. Such dosage and administration instructions can be of the kind that is provided to a doctor, or they can be of the kind that are provided by a doctor, such as instructions to a patient.

### Compositions

Therefore, according to the present disclosure, the BTN3A activating antibody as herein defined and the Bcl2 family inhibitor as herein defined can be, for example, formulated individually into separate compositions with pharmaceutically acceptable carriers, e.g., pharmaceutical compositions.

As used herein, “pharmaceutically acceptable carrier” refers to a diluent, adjuvant or excipient and includes any and all solvents, dispersion media, coatings, antibacterial and antifungal agents, isotonic and absorption delaying agents, and the like that are physiologically compatible. The composition may further comprise one or more of the following compounds in addition to the active compound (i.e., the BTN3A activating antibody and/or the Bcl2 family inhibitor).

Sterile phosphate-buffered saline is one example of a pharmaceutically acceptable carrier. Other suitable carriers are well-known to those in the art. (Remington and Gennaro, 1995) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, parenteral, intranasal, intravenous, intramuscular, subcutaneous, or intraocular administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

### Compositions Comprising a BTN3A Activating Antibody and Dosage Regimen

In some embodiments the BTN3A activating antibody as herein defined may thus be formulated in a composition, e.g., a pharmaceutical composition as defined above, containing one or a combination of antibodies disclosed herein, for example, one antibody selected from the group consisting of mAb1, mAb2, mAb3, mAb4 and mAb5 or their antigen-binding portions, formulated together with a pharmaceutically acceptable carrier.

An antibody of the disclosure can be formulated into a composition as above defined in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Suitable formulation for solution for infusion or subcutaneous injection of antibodies have been described in the art and, for example, are reviewed in Cui et al. (Drug Dev Ind Pharm 2017, 43(4): 519-530). In preferred embodiments, the anti-BTN3A antibody is formulated for intravenous infusion as above defined.

The pharmaceutical composition comprising the BTN3A activating antibody can be formulated at various concentrations. For example, the formulation may comprise the activating BTN3A activating antibody at a concentration of between 0.1 μM and 1 mM, more preferably between 1 μM and 500 μM, between 500 μM and 1 mM, between 300 μM and 700 μM, between 1 μM and 200 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM, or between 900 μM and 1 mM. Typically, the formulation comprises the BTN3A activating antibody at a concentration of between 300 μM and 700 μM.

Typically, the therapeutic dose of the activating BTN3A activating antibody in a human patient will be in the range of 100 μg to 700 mg per administration (based on a body weight of 70 kg). For example, the maximum therapeutic dose may be in the range of 0.1 to 10 mg/kg per administration, e.g., between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly, every three weeks or monthly.

Typically, in specific embodiments, said activating BTN3A activating antibody is administered intravenously at a dose comprised between 20 μg and 200 mg, notably between 1 mg and 200 mg or between 7 and 200 mg each dose, typically every 21 days.

In specific embodiments, suitable dose for intravenous administration of an activating anti-BTN3A antibody can be selected from 1, 7, 10, 20, 50, 75, 100, 125, 150, 175 and 200 mg.

In specific embodiments, the activating BTN3A activating antibody for use according to the methods of the disclosure (preferably mAb1 as described hereafter) is administered intravenously at a dose comprised between 20 μg and 200 mg each dose, preferably a second dose being administered at least 15 days after the first dose, typically after about 21 days.

### Compositions Comprising a Bcl2 Family Inhibitor and Dosage Regimen

Therapy with Bcl2 family inhibitors may be initiated according to a weekly ramp-up schedule over a specific period of several days or weeks to the recommended daily dose.

For treating chronic lymphocytic leukemia (CLL) and small lymphocytic lymphoma (SLL), Venetoclax is currently administered at a daily dose of 20 mg for Week 1, a daily dose of 50 mg for Week 2, a daily dose of 100 mg for Week 3, a daily dose of 200 mg for Week 4, and a daily dose of 400 mg for Week 5 and beyond. For treating AML in combination therapy with another agent, such as injectable 5-Azacytidine, Venetoclax is currently administered at a daily dose of 100 mg for Day 1, a daily dose of 200 mg for Day 2, and a daily dose of 400 mg for Days 3 and beyond. VIDAZA® (5-Zzacytidine for injection) is administered in 28-day cycles, beginning on Day 1 of Venetoclax treatment, at a dosage of 75 mg/m² intravenously or subcutaneously, on Days 1-7 of each cycle.

In some embodiments, Venetoclax is administered orally. In some embodiments, Venetoclax is administered in a form of a tablet. In some embodiments, Venetoclax is administered daily. In some embodiments, Venetoclax is administered at a dose of from about 20 mg to about 400 mg, such as about 20 mg, about 50 mg, about 100 mg, about 200 mg, or about 400 mg. In some embodiments, Venetoclax is administered at a dose of about 400 mg.

In some embodiments, 5-Azacytidine and Venetoclax are administered concomitantly. In some embodiments, 5-Azacytidine and Venetoclax are administered sequentially. In some embodiments, where the 5-Azacytidine and Venetoclax are administered sequentially, the 5-Azacytidine is administered first. In some embodiments, 5-Azacytidine and Venetoclax are administered as separate dosage forms, such as injections suitable for intravenous or subcutaneous use and/or tablets or capsules for oral use. In some embodiments, 5-Azacytidine and Venetoclax are co-formulated as a single unit dosage form, such as an injection suitable for intravenous or subcutaneous use or a tablet or capsule for oral use.

Other specific dosage regimen with other Bcl2 family inhibitor may be determined by the skilled person in the art, in particular with the respect to the prescribed regimen for the therapeutic indications to be treated by the combined therapy of the present disclosure.

### Uses and Methods of the Combination of the Disclosure

The present disclosure provides a therapeutic combination comprising a BTN3A activating antibody, e.g., mAb1, and a Bcl2 family inhibitor, for example, a Bcl2 inhibitor, e.g., Venetoclax, and optionally a hypomethylating agent, e.g., Azacytidine; as previously defined for use in the treatment of cancer, in particular hematological malignancies, and more preferably acute myeloid leukemia.

The present disclosure also provides a method of treatment of cancer in a patient in need thereof comprising administering to said patient a therapeutically effective amount of a BTN3A activating antibody, e.g., mAb1, in combination, simultaneously, sequentially, or separately with a therapeutically effective amount of Bcl2 family inhibitor, for example, a Bcl2 inhibitor, e.g., Venetoclax, and, optionally with a therapeutically effective amount of a hypomethylating agent such as Azacytidine or Decitabine.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of acute myeloid leukemia, the term “treatment” may refer to the inhibition of the proliferation of AML blasts, or the reduction of the number of AML blasts.

The antibodies of the disclosure are BTN3A activating antibodies and can activate the cytolytic function, cytokine production and/or proliferation of Vγ9Vδ2 T cells, and thereby may be used to overcome the immunosuppressive mechanisms observed in cancer patients (see notably WO2012/080769, WO2012/080351, and WO2020/025703) and during chronic infections. The results of the present disclosure now show that the present combination promotes further synergistic and specific Vγ9Vδ2 T cell killing towards AML blasts in human PBMCs highlighting its therapeutic interest, notably for the treatment of hematological malignancies.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

Examples of cancers include, but are not limited to, hematological malignancies such as a leukemia (e.g., an acute myeloid leukemia (AML) or a chronic lymphocytic leukemia (CLL) including chronic B cell leukemia), a lymphoma (e.g., a small lymphocytic lymphoma (SLL)), diffuse large B cell lymphoma (DLBCL), follicular lymphoma, or a myeloma (e.g., a multiple myeloma (MM)). In some embodiments, the hematological cancer is a myelodysplastic syndrome (MDS) (e.g., a lower risk MDS, e.g., a very low risk MDS, a low risk MDS, or an intermediate risk MDS, or a higher risk myelodysplastic syndrome, e.g., a high risk MDS or a very high risk MDS).

In some embodiments, the subject in need of such treatment is a subject suffering from hematological malignancies, e.g., acute myeloid leukemia, who is not eligible for use of intensive induction chemotherapy.

In some embodiments, the subject in need of such treatment is 75 years or older, and/or has comorbidities.

In some embodiments, the subject in need of such treatment is a subject who is indicated for a treatment with Venetoclax in combination with hypomethylating agents according to the standard of care.

Each therapeutic agent (i.e., the BTN3A activating antibody, the Bcl2 inhibitor and optionally, the hypomathylating agent, as previously defined) in the combination therapy of the present disclosure may be administered in separate medicaments administered sequentially in any order. The anti-BTN3A antibody, e.g. mAb1, the Bcl2 family inhibitor, for example a Bcl2 inhibitor such as Venetoclax, and the hypomethylating agent, e.g., Azacytidine, are typically formulated into separate compositions as previously described.

Sequential administration (e.g., in separate pharmaceutical compositions) is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the BTN3A activating antibody (e.g., mAb1) is administered before first administration of the Bcl2 family inhibitor, for example a Bcl2 inhibitor (e.g., Venetoclax), while in other embodiments the BTN3A activating antibody (e.g., mAb1) is administered after administration of the Bcl2 family inhibitor, for example a Bcl2 inhibitor (e.g., Venetoclax).

Selecting a dosage regimen (also referred to herein as an administration regimen) for a combination therapy of the disclosure depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules is available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Any suitable dosage range may be used as determined by attending medical personnel. Dosage regimens can be adjusted to provide the optimum desired response (e.g., a therapeutic or prophylactic response). Dosages have been disclosed in the previous section related to Bcl2 family inhibitor. In some embodiments of the combination therapy, a suitable dosage range for the Bcl2 family inhibitor and notably for Venetoclax as herein defined may, for instance, be 10 mg-600 mg. In some embodiments, exemplary doses are from about 100 mg to 400 mg. In some embodiments, administration regimens include providing the Bcl2 family inhibitor, for example a Bcl2 inhibitor, e.g. Venetoclax, on a daily basis.

In some embodiments, at least one of the therapeutic agents in the combination therapy is administered using the same dosage regimen (dose, frequency and duration of treatment) that is typically employed when the agent is used as monotherapy for treating the same cancer.

For example, Venetoclax is administered at the same dosage regimen as the one recommended for treating newly diagnosed patients with acute myeloid leukemia in combination with hypomethylating agents such as Azacytidine (see “Practical Dosing Considerations for Venetoclax”, Cancernetwork.com, Oncology, Vol 33, No. 9, Volume 33, issue 9).

When the Bcl2 family inhibitor, for example a Bcl2 inhibitor, e.g., Venetoclax, is administered in combination with a hypomethylating agent (e.g., Azacytidine), the hypomethylating agent may be administered once a day at 75 mg/m² subcutaneously on days 1-5 and 8-9 of each cycle. Between cycles, up to 14 days of recovery are recommended in case of lymphopenia (Jonas and Pollyea, 2019; Leukemia 33: 2795-2804) with an administration of growth factors to at least treat neutropenia. Following cycles are determined by patient response, but are quite similar to cycle 1.

In other embodiments, the patient receives a lower total amount of at least one of the therapeutic agents in the combination therapy than when the agent is used as monotherapy, e.g., smaller doses, less frequent doses, and/or shorter treatment duration.

Regarding the dosage regimen of the BTN3A activating antibody for use in the present therapeutic combination any suitable dosage range may be used as determined by attending medical personnel.

Dosage regimens can be adjusted to provide the optimum desired response (e.g., a therapeutic or prophylactic response). The antibodies of the disclosure may be formulated within a therapeutic mixture to comprise about 1 to 200.0 milligrams. It will be appreciated that such a dose may be administered at different intervals, as determined by the oncologist/physician; for example, a dose may be administered daily, twice-weekly, weekly, bi-weekly, every three weeks or monthly.

Typically, in specific embodiments, said BTN3A activating antibody, e.g., mAb1, is administered intravenously at a dose comprised between 1 mg and 200 mg each dose, for example between 20 and 100 mg, typically every 21 days. In specific embodiments, suitable dose for intravenous administration of an activating BTN3A antibody, e.g., mAb1, can be selected from 1, 7, 10, 20, 50, 75, 100, 125, 150, 175 and 200 mg.

In specific embodiments, the activating BTN3A antibody for use according to the combination methods of the disclosure (preferably mAb1 as described hereafter) is administered intravenously at a dose comprised between 7 mg and 200 mg each dose, preferably a second dose being administered at least 15 days after the first dose, typically after once every 21 days, for 1 to 22 cycles.

In some embodiments, the Bcl-2 family inhibitor, for example a Bcl-2 inhibitor, e.g. Venetoclax, is administered in combination, separately, sequentially, or simultaneously, with at a dose of about 10 mg to about 500 mg, e.g., about 20 mg to about 400 mg, about 50 mg to about 350 mg, about 100 mg to about 300 mg, about 150 mg to about 250 mg, 50 mg to about 500 mg, about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, about 450 mg to about 500 mg, about 10 mg to about 400 mg, about 10 mg to about 350 mg, about 10 mg to 300 mg, about 10 mg to about 250 mg, about 10 mg to about 200 mg, about 10 mg to about 150 mg, about 10 mg to about 100 mg, about 10 mg to about 50 mg, about 50 mg to about 150 mg, about 150 mg to about 250 mg, about 250 mg to about 350 mg, or about 350 mg to about 400 mg. In some embodiments, the Bcl-2 inhibitor, e.g. Venetoclax, is administered at a dose of about 20 mg, 50 mg, 100 mg, 150 mg, 200 mg, 250 mg, 300 mg, 350 mg, 400 mg, 450 mg, or 500 mg. In some embodiments, the Bcl-2 inhibitor, e.g. Venetoclax, is administered once a day. In some embodiments, the Bcl-2 family inhibitor, for example a Bcl-2 inhibitor, is administered orally.

In some embodiments, the Bcl-2 family inhibitor, for example a Bcl-2 inhibitor, e.g. Venetoclax, is administered at a dose of about 350 mg to about 450 mg (e.g., about 400 mg) orally, once a day, e.g., on each day of a 21-day cycle. In some embodiments, the dose of the Bcl-2 inhibitor is ramped-up over a period of 4 days in the first cycle to achieve the dose of about 400 mg per day. For example, the doses for Cycle 1 Day 1, Day 2, Day 3, and Day 4 and beyond are about 100 mg, about 200 mg, about 300 mg, and about 400 mg, respectively.

In some embodiments, the Bcl-2 family inhibitor, for example a Bcl-2 inhibitor, e.g. Venetoclax is administered in a ramp-up cycle for e.g., about 5 weeks, followed by fixed dose for e.g., at least about 24 months. In some embodiments, the Bcl-2 inhibitor is administered at a dose of about 10 mg to about 30 mg (e.g., about 20 mg) once a day for e.g., about 1 week, followed by about 40 mg to about 60 mg (e.g., about 50 mg) once a day for e.g., about 1 week, followed by about 80 mg to about 120 mg (e.g., about 100 mg) once a day for e.g., about 1 week, followed by about 150 mg to about 250 mg (e.g., about 200 mg) once a day for e.g., about 1 week, followed by about 350 mg to about 450 mg (e.g., about 400 mg) once a day for e.g., about 1 week, and followed by a fixed dose, e.g., about 350 mg to about 450 mg (e.g., about 400 mg), once a day, for e.g., at least about 24 months.

In specific embodiments, the Bcl2 family inhibitor, for example a Bcl-2 inhibitor, e.g., Venetoclax, is administered once a day, for example at a unit dose between 10 mg and 600 mg, optionally in combination with Azacytidine or Decitabine, for at least 5 to 9 days, followed by a recovery period of at least 14 days.

In specific embodiments, the Bcl2 family inhibitor, for example a Bcl-2 inhibitor, e.g., Venetoclax, and the hypomethylating agent, e.g., Azacytidine, are first administered for at least one cycle followed by a recovery period of at least 10 to 14 days, and said BTN3A activating antibody is administered for example, after at least 10 to 14 days of said recovery period, or along with cycle 2 of administration of said Bcl2 inhibitor and hypomethylating agent.

In other specific embodiments, the first administration of the Bcl2 family inhibitor, for example a Bcl-2 inhibitor, e.g., Venetoclax, and the hypomethylating agent, e.g. Azacytidine, occurs after a first cycle of treatment with BTN3A activating antibody, e.g., mAb1, preferably after 21 days following the first administration of said BTN3A activating antibody.

In specific embodiments, the activating BTN3A antibody for use according to the methods of the disclosure (preferably mAb1 as described hereafter) is administered intravenously at a dose comprised between 7 mg and 200 mg each dose, preferably a second dose being administered at least 15 days after the first dose, typically after once every 21 days, for 1 to 22 cycles; and the Bcl2 family inhibitor, for example a Bcl-2 inhibitor, e.g., Venetoclax, is administered once a day, for example at a unit dose between 10 mg and 600 mg, optionally in combination with Azacytidine or Decitabine, for at least 5 to 9 days, followed by a recovery period of at least 14 days, and wherein Venetoclax, and the hypomethylating agent, e.g.

Azacytidine, are first administered for at least one cycle followed by a recovery period of at least 10 to 14 days, and said BTN3A activating antibody is administered for example, after at least 10 to 14 days of said recovery period.

In specific embodiments, the combination therapy as herein disclosed (typically mAb1 as previously described and Bcl2 family inhibitor, for example a Bcl-2 inhibitor, notably Venetoclax) may be administered in combination with other anti-neoplastic agents.

In other specific embodiments, the combination therapy (typically mAb1 as previously described and said Bcl2 family inhibitor, for example a Bcl-2 inhibitor, notably Venetoclax) as herein disclosed may be administered in combination with cell therapy (in particular γδT cell therapy).

The disclosure thus relates to the combination as herein defined for use in vivo to potentiate tumor cells in a γδ T cell therapy in a subject in need thereof, typically suffering from cancer, wherein the BTN3A activating antibody and the Bcl2 family inhibitor for example a Bcl-2 inhibitor, can be administered concomitantly, simultaneously, concurrently or sequentially to the subject.

As used herein, the term γδ T cell therapy refers to a therapy which comprises the administration to a subject in need thereof of at least an efficient amount of γδ T cells. Such γδ T cells may be allogeneic or autologous. In specific embodiments, the γδ T cells can be genetically engineered by deletion or knock-out or insertion or knock-in of specific genes. In specific embodiments, said γδ T cells include γδ T cells expressing chimeric antigen receptor. The γδ T cells may have been expanded and/or purified ex vivo. Alternatively, the γδ T cells may also be comprised in a cell composition comprising other blood cells, and for example other cells of the immune system. For references regarding γδ T cell therapy, please see Pauza C D. et al., Front Immunol. 2018 Jun. 8; 9:1305. doi: 10.3389; Saudemont A. et al., Front Immunol. 2018 Feb. 5; 9:153. doi: 10.3389.

The disclosure thus relates to a method of treatment of a subject suffering from cancer including solid tumors or hematological malignancies, in particular, leukemias such as acute myeloid leukemia, and having tumor cells, for example blood tumor cells, said method comprising:
(i) administering in said subject an efficient amount of anti-BTN3A activating antibodies as disclosed herein, typically mAb1, mAb2, mAb3, mAb4 or mAb5 in combination with an efficient amount of Bcl2 inhibitor, e.g. Venetoclax, and optionally, a hypomethylating agent, e.g. Azacytidine, and,
(ii) administering an efficient amount of γδ T cell composition in said subject,
   wherein the combination of said efficient amount of ant-BTN3A antibodies and said efficient amount of Bcl2 inhibitor has the capacity to potentiate antitumor cytolysis mediated by said γδ T cell composition against said tumor cells.

The disclosure having been fully described is now further illustrated by the following examples, which are illustrative only and are not meant to be further limiting.

TABLE 4
Sequences used in the present disclosure

| SEQ ID | | Description of the | |
|---|---|---|---|
| NO: | Type | sequence | sequence |
| 1 | aa | Humanized heavy | QVQLVQSGAEVKKPGASVKLSCKASGYIFTRYY |
| | | chain variable region | MYWVKQRPGQGLEWIGEINPNNGGTKFNEKFK |
| | | VH2 of mAb 7.2 | NRATLTVDKSISTAYMELSRLRSDDTAVYYCSR |
| | | | EDDYDGTPFAMDYWGQGTLVTVSS |
| 2 | aa | Humanized light chain | DIQMTQSPSSLSASVGDRVTITCHASQNINVWL |
| | | variable region Vk1 of | SWYQQKPGKAPKLLIYKASNLHTGVPSRFTGSG |
| | | mAb 7.2 | SGTDFTFTISSLQPEDIATYYCQQGQTYPYTFGQ |
| | | | GTKLEIK |
| 3 | aa | Humanized light chain | DIQMTQSPSSLSASVGDRVTITCHASQNINVWL |
| | | variable region Vk2 of | SWYQQKPGKAPKLLIYKASNLHTGVPSRFSGSG |
| | | mAb 7.2 | SGTDFTFTISSLQPEDIATYYCQQGQTYPYTFGQ |
| | | | GTKLEIK |
| 4 | aa | Full length heavy chain | QVQLVQSGAEVKKPGASVKLSCKASGYIFTRYY |
| | | of mAbs 1 and 2 (VH2 | MYWVKQRPGQGLEWIGEINPNNGGTKFNEKFK |
| | | 7.2 silent IgG1) | NRATLTVDKSISTAYMELSRLRSDDTAVYYCSR |
| | | | EDDYDGTPFAMDYWGQGTLVTVSSASTKGPSV |
| | | | FPLAPSSKSTSGGTAALGCLVKDYFPEPVTVSW |
| | | | NSGALTSGVHTFPAVLQSSGLYSLSSVVTVPSS |
| | | | SLGTQTYICNVNHKPSNTKVDKRVEPKSCDKTH |
| | | | TCPPCPAPEFEGGPSVFLFPPKPKDTLMISRTPE |
| | | | VTCVVVDVSHEDPEVKFNWYVDGVEVHNAKTK |
| | | | PREEQYNSTYRVVSVLTVLHQDWLNGKEYKCK |
| | | | VSNKALPASIEKTISKAKGQPREPQVYTLPPSRE |
| | | | EMTKNQVSLTCLVKGFYPSDIAVEWESNGQPE |
| | | | NNYKTTPPVLDSDGSFFLYSKLTVDKSRWQQG |
| | | | NVFSCSVMHEALHNHYTQKSLSLSPG |
| 5 | aa | Full length heavy chain | QVQLVQSGAEVKKPGASVKLSCKASGYIFTRYY |
| | | of mAbs 4 and 5 (VH2 | MYWVKQRPGQGLEWIGEINPNNGGTKFNEKFK |
| | | 7.2 silent IgG4) | NRATLTVDKSISTAYMELSRLRSDDTAVYYCSR |
| | | | EDDYDGTPFAMDYWGQGTLVTVSSASTKGPSV |
| | | | FPLAPCSRSTSESTAALGCLVKDYFPEPVTVSW |
| | | | NSGALTSGVHTFPAVLQSSGLYSLSSVVTVPSS |
| | | | SLGTKTYTCNVDHKPSNTKVDKRVESKYGPPCP |
| | | | PCPAPEFEGGPSVFLFPPKPKDTLMISRTPEVTC |
| | | | VVVDVSQEDPEVQFNWYVDGVEVHNAKTKPRE |
| | | | EQFNSTYRVVSVLTVLHQDWLNGKEYKCKVSN |
| | | | KGLPSSIEKTISKAKGQPREPQVYTLPPSQEEMT |
| | | | KNQVSLTCLVKGFYPSDIAVEWESNGQPENNYK |
| | | | TTPPVLDSDGSFFLYSRLTVDKSRWQEGNVFSC |
| | | | SVMHEALHNHYTQKSLSLSLG |
| 6 | aa | Full length light chain of | DIQMTQSPSSLSASVGDRVTITCHASQNINVWL |
| | | mAbs 1 and 4 (Vk1 7.2) | SWYQQKPGKAPKLLIYKASNLHTGVPSRFTGSG |
| | | | SGTDFTFTISSLQPEDIATYYCQQGQTYPYTFGQ |
| | | | GTKLEIKRTVAAPSVFIFPPSDEQLKSGTASVVC |
| | | | LLNNFYPREAKVQWKVDNALQSGNSQESVTEQ |
| | | | DSKDSTYSLSSTLTLSKADYEKHKVYACEVTHQ |
| | | | GLSSPVTKSFNRGEC |
| 7 | aa | Full length light chain of | DIQMTQSPSSLSASVGDRVTITCHASQNINVWL |
| | | mAbs 2 and 5 (Vk2 7.2) | SWYQQKPGKAPKLLIYKASNLHTGVPSRFSGSG |
| | | | SGTDFTFTISSLQPEDIATYYCQQGQTYPYTFGQ |
| | | | GTKLEIKRTVAAPSVFIFPPSDEQLKSGTASVVC |
| | | | LLNNFYPREAKVQWKVDNALQSGNSQESVTEQ |
| | | | DSKDSTYSLSSTLTLSKADYEKHKVYACEVTHQ |
| | | | GLSSPVTKSFNRGEC |
| 8 | nt | Full length heavy chain | CAGGTCCAACTGGTGCAGTCTGGGGCTGAAG |
| | | of mAbs 1 and 2 (VH2 | TGAAGAAGCCTGGGGCTTCAGTGAAGTTGTC |
| | | 7.2 silent IgG1) | CTGCAAGGCTTCTGGCTACATCTTCACCAGAT |
| | | | ACTATATGTATTGGGTGAAGCAGAGGCCTGGA |
| | | | CAAGGCCTTGAGTGGATTGGAGAGATTAATCC |
| | | | TAACAATGGTGGTACTAAGTTCAATGAGAAGT |
| | | | TCAAGAACAGGGCCACACTGACTGTAGACAAA |
| | | | TCCATCAGCACAGCATACATGGAGCTCAGCA |
| | | | GGCTGAGATCTGACGACACGGCGGTCTATTA |
| | | | TTGTTCAAGAGAGGATGATTACGACGGGACC |
| | | | CCCTTTGCTATGGACTACTGGGGTCAAGGAAC |
| | | | CCTGGTCACCGTCTCCTCAGCCTCCACCAAG |
| | | | GGCCCATCGGTCTTCCCCCTGGCACCCTCCT |
| | | | CCAAGAGCACCTCTGGGGGCACAGCGGCCCT |
| | | | GGGCTGCCTGGTCAAGGACTACTTCCCCGAA |
| | | | CCGGTGACGGTGTCGTGGAACTCAGGCGCCC |
| | | | TGACCAGCGGCGTGCACACCTTCCCGGCTGT |
| | | | CCTACAGTCCTCAGGACTCTACTCCCTCAGCA |
| | | | GCGTGGTGACCGTGCCCTCCAGCAGCTTGGG |
| | | | CACCCAGACCTACATCTGCAACGTGAATCACA |
| | | | AGCCCAGCAACACCAAGGTGGACAAGAGAGT |
| | | | TGAGCCCAAATCTTGTGACAAAACTCACACAT |
| | | | GCCCACCGTGCCCAGCACCTGAATTCGAGGG |
| | | | GGGACCGTCAGTCTTCCTCTTCCCCCCAAAAC |
| | | | CCAAGGACACCCTCATGATCTCCCGGACCCC |
| | | | TGAGGTCACATGCGTGGTGGTGGACGTGAGC |
| | | | CACGAAGACCCTGAGGTCAAGTTCAACTGGTA |
| | | | CGTGGACGGCGTGGAGGTGCATAATGCCAAG |
| | | | ACAAAGCCGCGGGAGGAGCAGTACAACAGCA |
| | | | CGTACCGTGTGGTCAGCGTCCTCACCGTCCT |
| | | | GCACCAGGACTGGCTGAATGGCAAGGAGTAC |
| | | | AAGTGCAAGGTCTCCAACAAAGCCCTCCCAG |
| | | | CCTCCATCGAGAAAACCATCTCCAAAGCCAAA |
| | | | GGGCAGCCCCGAGAACCACAGGTGTACACCC |
| | | | TGCCCCCATCCCGGGAAGAGATGACCAAGAA |
| | | | CCAGGTCAGCCTGACCTGCCTGGTCAAAGGC |
| | | | TTCTATCCCAGCGACATCGCCGTGGAGTGGG |
| | | | AGAGCAATGGGCAGCCGGAGAACAACTACAA |
| | | | GACCACGCCTCCCGTGCTGGACTCCGACGGC |
| | | | TCCTTCTTCCTCTATAGCAAGCTCACCGTGGA |
| | | | CAAGAGCAGGTGGCAGCAGGGGAACGTCTTC |
| | | | TCATGCTCCGTGATGCATGAGGCTCTGCACAA |
| | | | CCACTACACGCAGAAGAGCCTCTCCCTGTCTC |
| | | | CGGGTTGA |
| 9 | nt | Full length heavy chain | CAGGTCCAACTGGTGCAGTCTGGGGCTGAAG |
| | | of mAbs 4 and 5 (VH2 | TGAAGAAGCCTGGGGCTTCAGTGAAGTTGTC |
| | | 7.2 silent IgG4) | CTGCAAGGCTTCTGGCTACATCTTCACCAGAT |
| | | | ACTATATGTATTGGGTGAAGCAGAGGCCTGGA |
| | | | CAAGGCCTTGAGTGGATTGGAGAGATTAATCC |
| | | | TAACAATGGTGGTACTAAGTTCAATGAGAAGT |
| | | | TCAAGAACAGGGCCACACTGACTGTAGACAAA |
| | | | TCCATCAGCACAGCATACATGGAGCTCAGCA |
| | | | GGCTGAGATCTGACGACACGGCGGTCTATTA |
| | | | TTGTTCAAGAGAGGATGATTACGACGGGACC |
| | | | CCCTTTGCTATGGACTACTGGGGTCAAGGAAC |
| | | | CCTGGTCACCGTCTCCTCAGCTTCCACCAAG |
| | | | GGCCCATCCGTCTTCCCCCTGGCGCCCTGCT |
| | | | CCAGGAGCACCTCCGAGAGCACAGCCGCCCT |
| | | | GGGCTGCCTGGTCAAGGACTACTTCCCCGAA |
| | | | CCGGTGACGGTGTCGTGGAACTCAGGCGCCC |
| | | | TGACCAGCGGCGTGCACACCTTCCCGGCTGT |
| | | | CCTACAGTCCTCAGGACTCTACTCCCTCAGCA |
| | | | GCGTGGTGACCGTGCCCTCCAGCAGCTTGGG |
| | | | CACGAAGACCTACACCTGCAATGTAGATCACA |
| | | | AGCCCAGCAACACCAAGGTGGACAAGAGAGT |
| | | | TGAGTCCAAATATGGTCCCCCATGCCCACCAT |
| | | | GCCCAGCACCTGAGTTCGAGGGGGGACCATC |
| | | | AGTCTTCCTGTTCCCCCCAAAACCCAAGGACA |
| | | | CTCTCATGATCTCCCGGACCCCTGAGGTCAC |
| | | | GTGCGTGGTGGTGGACGTGAGCCAGGAAGAC |
| | | | CCCGAGGTCCAGTTCAACTGGTACGTGGATG |
| | | | GCGTGGAGGTGCATAATGCCAAGACAAAGCC |
| | | | GCGGGAGGAGCAGTTCAACAGCACGTACCGT |
| | | | GTGGTCAGCGTCCTCACCGTCCTGCACCAGG |
| | | | ACTGGCTGAACGGCAAGGAGTACAAGTGCAA |
| | | | GGTCTCCAACAAAGGCCTCCCGTCCTCCATC |
| | | | GAGAAAACCATCTCCAAAGCCAAAGGGCAGC |
| | | | CCCGAGAGCCACAGGTGTACACCCTGCCCCC |
| | | | ATCCCAGGAGGAGATGACCAAGAACCAGGTC |
| | | | AGCCTGACCTGCCTGGTCAAAGGCTTCTACC |
| | | | CCAGCGACATCGCCGTGGAGTGGGAGAGCAA |
| | | | TGGGCAGCCGGAGAACAACTACAAGACCACG |
| | | | CCTCCCGTGCTGGACTCCGACGGCTCCTTCT |
| | | | TCCTCTACAGCAGGCTAACCGTGGACAAGAG |
| | | | CAGGTGGCAGGAGGGGAATGTCTTCTCATGC |
| | | | TCCGTGATGCATGAGGCTCTGCACAACCACTA |
| | | | CACACAGAAGAGCCTCTCCCTGTCTCTGGGTT |
| | | | GA |
| 10 | nt | Full length light chain of | GACATCCAGATGACCCAGTCTCCATCCAGTCT |
| | | mAbs 1 and 4 (Vk1 7.2) | GTCTGCATCCGTAGGAGACAGAGTCACCATC |
| | | | ACTTGCCATGCCAGTCAGAACATTAATGTTTG |
| | | | GTTATCTTGGTACCAGCAGAAACCAGGAAAAG |
| | | | CCCCTAAACTCTTGATCTATAAGGCTTCCAAC |
| | | | TTGCACACAGGCGTCCCATCAAGATTTACTGG |
| | | | CAGTGGATCTGGAACAGATTTCACATTCACCA |
| | | | TCAGCAGCCTGCAGCCTGAAGACATTGCCAC |
| | | | TTACTACTGTCAACAGGGTCAAACTTATCCATA |
| | | | CACGTTCGGACAGGGGACCAAGCTGGAGATC |
| | | | AAACGAACTGTGGCTGCACCATCTGTCTTCAT |
| | | | CTTCCCGCCATCTGATGAGCAGTTGAAATCTG |
| | | | GAACTGCCTCTGTTGTGTGCCTGCTGAATAAC |
| | | | TTCTATCCCAGAGAGGCCAAAGTACAGTGGAA |
| | | | GGTGGATAACGCCCTCCAATCGGGTAACTCC |
| | | | CAGGAGAGTGTCACAGAGCAGGACAGCAAGG |
| | | | ACAGCACCTACAGCCTCAGCAGCACCCTGAC |
| | | | GCTGAGCAAAGCAGACTACGAGAAACACAAA |
| | | | GTCTACGCCTGCGAAGTCACCCATCAGGGCC |
| | | | TGAGCTCGCCCGTCACAAAGAGCTTCAACAG |
| | | | GGGAGAGTGTTAG |
| 11 | nt | Full length light chain of | GACATCCAGATGACCCAGTCTCCATCCAGTCT |
| | | mAbs 2 and 5 (Vk2 7.2) | GTCTGCATCCGTAGGAGACAGAGTCACCATC |
| | | | ACTTGCCATGCCAGTCAGAACATTAATGTTTG |
| | | | GTTATCTTGGTACCAGCAGAAACCAGGAAAAG |
| | | | CCCCTAAACTCTTGATCTATAAGGCTTCCAAC |
| | | | TTGCACACAGGCGTCCCATCAAGATTTAGTGG |
| | | | CAGTGGATCTGGAACAGATTTCACATTCACCA |
| | | | TCAGCAGCCTGCAGCCTGAAGACATTGCCAC |
| | | | TTACTACTGTCAACAGGGTCAAACTTATCCATA |
| | | | CACGTTCGGACAGGGGACCAAGCTGGAGATC |
| | | | AAACGAACTGTGGCTGCACCATCTGTCTTCAT |
| | | | CTTCCCGCCATCTGATGAGCAGTTGAAATCTG |
| | | | GAACTGCCTCTGTTGTGTGCCTGCTGAATAAC |
| | | | TTCTATCCCAGAGAGGCCAAAGTACAGTGGAA |
| | | | GGTGGATAACGCCCTCCAATCGGGTAACTCC |
| | | | CAGGAGAGTGTCACAGAGCAGGACAGCAAGG |
| | | | ACAGCACCTACAGCCTCAGCAGCACCCTGAC |
| | | | GCTGAGCAAAGCAGACTACGAGAAACACAAA |
| | | | GTCTACGCCTGCGAAGTCACCCATCAGGGCC |
| | | | TGAGCTCGCCCGTCACAAAGAGCTTCAACAG |
| | | | GGGAGAGTGTTAG |
| 12 | aa | HCDR1 of mAb 7.2, 1, | RYYMY |
| | | 2, 4 and 5 | |
| 13 | aa | HCDR2 of mAb 7.2, 1, | EINPNNGGTKFNEKFKN |
| | | 2, 4 and 5 | |
| 14 | aa | HCDR3 of mAb 7.2, 1, | EDDYDGTPFAMDY |
| | | 2, 4 and 5 | |
| 15 | aa | LCDR1 of mAb 7.2, 1, | HASQNINVWLS |
| | | 2, 4 and 5 | |
| 16 | aa | LCDR2 of mAb 7.2, 1, | KASNLHT |
| | | 2, 4 and 5 | |
| 17 | aa | LCDR3 of mAb 7.2, 1, | QQGQTYPYT |
| | | 2, 4 and 5 | |
| 18 | aa | HCDR1 of mAb 20.1 | RYYLY |
| 19 | aa | HCDR2 of mAb 20.1 | EINPNNGGTKFNEKFKS |
| 20 | aa | HCDR3 of mAb 20.1 | EDDYDGTPDAMDY |
| 21 | aa | LCDR1 of mAb 20.1 | HASQNINLWLS |
| 22 | aa | LCDR2 of mAb 20.1 | RASNLHT |
| 23 | aa | LCDR3 of mAb 20.1 | QQGHSYPYT |
| 24 | aa | Human BTN3A1 | MKMASFLAFLLLNFRVCLLLLQLLMPHSAQFSVL |
| | | | GPSGPILAMVGEDADLPCHLFPTMSAETMELKW |
| | | | VSSSLRQVVNVYADGKEVEDRQSAPYRGRTSIL |
| | | | RDGITAGKAALRIHNVTASDSGKYLCYFQDGDF |
| | | | YEKALVELKVAALGSDLHVDVKGYKDGGIHLEC |
| | | | RSTGWYPQPQIQWSNNKGENIPTVEAPVVADG |
| | | | VGLYAVAASVIMRGSSGEGVSCTIRSSLLGLEKT |
| | | | ASISIADPFFRSAQRWIAALAGTLPVLLLLLGGAG |
| | | | YFLWQQQEEKKTQFRKKKREQELREMAWSTM |
| | | | KQEQSTRVKLLEELRWRSIQYASRGERHSAYNE |
| | | | WKKALFKPADVILDPKTANPILLVSEDQRSVQRA |
| | | | KEPQDLPDNPERFNWHYCVLGCESFISGRHYW |
| | | | EVEVGDRKEWHIGVCSKNVQRKGWVKMTPEN |
| | | | GFWTMGLTDGNKYRTLTEPRTNLKLPKPPKKV |
| | | | GVFLDYETGDISFYNAVDGSHIHTFLDVSFSEAL |
| | | | YPVFRILTLEPTALTICPA |
| 25 | aa | Human BTN3A2 | MKMASSLAFLLLNFHVSLLLVQLLTPCSAQFSVL |
| | | | GPSGPILAMVGEDADLPCHLFPTMSAETMELKW |
| | | | VSSSLRQVVNVYADGKEVEDRQSAPYRGRTSIL |
| | | | RDGITAGKAALRIHNVTASDSGKYLCYFQDGDF |
| | | | YEKALVELKVAALGSNLHVEVKGYEDGGIHLEC |
| | | | RSTGWYPQPQIQWSNAKGENIPAVEAPVVADG |
| | | | VGLYEVAASVIMRGGSGEGVSCIIRNSLLGLEKT |
| | | | ASISIADPFFRSAQPWIAALAGTLPILLLLLAGASY |
| | | | FLWRQQKEITALSSEIESEQEMKEMGYAATEREI |
| | | | SLRESLQEELKRKKIQYLTRGEESSSDTNKSA |
| 26 | aa | Human BTN3A3 | MKMASSLAFLLLNFHVSLFLVQLLTPCSAQFSVL |
| | | | GPSGPILAMVGEDADLPCHLFPTMSAETMELR |
| | | | WVSSSLRQVVNVYADGKEVEDRQSAPYRGRTS |
| | | | ILRDGITAGKAALRIHNVTASDSGKYLCYFQDGD |
| | | | FYEKALVELKVAALGSDLHIEVKGYEDGGIHLEC |
| | | | RSTGWYPQPQIKWSDTKGENIPAVEAPVVADG |
| | | | VGLYAVAASVIMRGSSGGGVSCIIRNSLLGLEKT |
| | | | ASISIADPFFRSAQPWIAALAGTLPISLLLLAGAS |
| | | | YFLWRQQKEKIALSRETEREREMKEMGYAATE |
| | | | QEISLREKLQEELKWRKIQYMARGEKSLAYHEW |
| | | | KMALFKPADVILDPDTANAILLVSEDQRSVQRAE |
| | | | EPRDLPDNPERFEWRYCVLGCENFTSGRHYWE |
| | | | VEVGDRKEWHIGVCSKNVERKKGWVKMTPEN |
| | | | GYWTMGLTDGNKYRALTEPRTNLKLPEPPRKV |
| | | | GIFLDYETGEISFYNATDGSHIYTFPHASFSEPLY |
| | | | PVFRILTLEPTALTICPIPKEVESSPDPDLVPDHS |
| | | | LETPLTPGLANESGEPQAEVTSLLLPAHPGAEV |
| | | | SPSATTNQNHKLQARTEALY |
| 27 | aa | Cynomolgus macaque | MGSSLAFLLLSFHVCVLLLQLLMPHSAQFAVVG |
| | | (m. fascicularis) | PPGPILAMVGEDADLPCHLFPTMSAETMELRWV |
| | | BTN3A1 ectodomain | SSNLRQVVNVYADGKEVEDRQSAAYRGRTSILR |
| | | used for recombinant | DGITAGKAALRIHNVTASDSGKYLCYFQDGDFY |
| | | protein production | EKALVELKVAALGSDLHIDVKGYEDGGIHLECRS |
| | | | TGWYPQPQIRWSNDKGENIPAVEAPVFVDGVG |
| | | | LYAVAASVILRGSSGEGVSCTIRSSLLGLEKTTSI |
| | | | SIAG |
| 28 | aa | Cynomolgus macaque | MGSSLAFLLLNFHVSFFLVQLLTPCSAQFSVLGP |
| | | (m. fascicularis) | SGPILAMVGEDADLPCHLFPTMSAETMELRWVS |
| | | BTN3A2 ectodomain | SSLRQVVNVYADGKEVEDRQSAPYRGRTSILRD |
| | | used for recombinant | DIAAGKAALRIHNVTASDSGKYLCYFQDADFYEK |
| | | protein production | ALVELKVAALGSNLHVEVKGYEDGGIHLECRST |
| | | | GWYPQPKIQWSNAKGQNIPAVEAPVVADGVGL |
| | | | YAVAASVIMRGGSGESVSCIIRNSVLGLEKTASIS |
| | | | IAD |
| 29 | aa | Cynomolgus macaque | MANFLAFLLLNFRVCLLLVQLLTPCSAQFAVLGP |
| | | (m. fascicularis) | HGPILAMVGEDVDLPCHLFPTMSAETMELRWVS |
| | | BTN3A3 ectodomain | SSLRQVVNVYSDGKEVEDRQSAPYRGRTSILRD |
| | | used for recombinant | GITAGKAALRIHNVTASDSGKYLCYFQDGDFYE |
| | | protein production | KALVELKVAALGSDLHIEVKGYEDGGIHLECRST |
| | | | GWYPQPQIQWSNTKGQHIPAVKAPVVADGVGL |
| | | | YAVAASVIMRGSSGEGVSCIIRNSLLGLEKTASIS |
| | | | ITD |
| 30 | aa | VH Humanized | QVQLVQSGAEVKKPGASVKVSCKASGYTFTRYYLY |
| | | mAb20.1 | WVKQRPGQGLEWIGEINPNNGGTKFNEKFKSRATM |
| | | | TVDKSTRTTYMELSSLRSEDTAVYYCSREDDYDGTP |
| | | | DAMDYWGQGTLVTVSS |
| 31 | aa | VL Humanized | DIQMTQSPSSLSASVGDRVTITCHASQNINLWLSWY |
| | | mAb20.1 | QQKPGKAPKLLIYRASNLHTGVPSRFSGSGSATDFTF |
| | | | TISSLQPEDIATYYCQQGHSYPYTFGGGTKVDIK |
| 32 | aa | Heavy chain | QVQLVQSGAEVKKPGASVKVSCKASGYTFTRYYLY |
| | | humanized mAb20.1 | WVKQRPGQGLEWIGEINPNNGGTKFNEKFKSRATM |
| | | | TVDKSTRTTYMELSSLRSEDTAVYYCSREDDYDGTP |
| | | | DAMDYWGQGTLVTVSSASTKGPSVFPLAPSSKSTS |
| | | | GGTAALGCLVKDYFPEPVTVSWNSGALTSGVHTFPA |
| | | | VLQSSGLYSLSSVVTVPSSSLGTQTYICNVNHKPSNT |
| | | | KVDKRVEPKSCDKTHTCPPCPAPEFEGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNGK |
| | | | EYKCKVSNKALPASIEKTISKAKGQPREPQVYTLPPS |
| | | | REEMTKNQVSLTCLVKGFYPSDIAVEWESNGQPENN |
| | | | YKTTPPVLDSDGSFFLYSKLTVDKSRWQQGNVFSCS |
| | | | VMHEALHNHYTQKSLSLSPGK |
| 33 | aa | Light chain humanized | DIQMTQSPSSLSASVGDRVTITCHASQNINLWLSWY |
| | | mAb20.1 | QQKPGKAPKLLIYRASNLHTGVPSRFSGSGSATDFTF |
| | | | TISSLQPEDIATYYCQQGHSYPYTFGGGTKVDIKRTV |
| | | | AAPSVFIFPPSDEQLKSGTASVVCLLNNFYPREAKVQ |
| | | | WKVDNALQSGNSQESVTEQDSKDSTYSLSSTLTLSK |
| | | | ADYEKHKVYACEVTHQGLSSPVTKSFNRGEC |
| 34 | aa | CDRH1 of variant | RYYLY |
| | | mAb20.1 | |
| 35 | aa | CDRH2 of variant | EINPSNGGTNFNEKFKS |
| | | mAb20.1 | |
| 36 | aa | CDRH3 of variant | EDDYDGTPDAMDY |
| | | mAb20.1 | |
| 37 | aa | CDRL1 of variant | HASQNINVWLS |
| | | mAb20.1 | |
| 38 | aa | CDRL2 of variant | KASNLHT |
| | | mAb20.1 | |
| 39 | aa | CDRL3 of variant | QGQSYPLT |
| | | mAb20.1 | |
| 40 | aa | VH of variant mAb20.1 | QVQLQQSGAELVKPGASVKLSCKASGYTFTRYYLYW |
| | | | VKQRPGQGLEWIGEINPSNGGTNFNEKFKSKATLTV |
| | | | DKSSRTTYIQLSSLTSEDSAVYYCSREDDYDGTPDA |
| | | | MDYWGQGTAVTVSS |
| 41 | aa | VL of variant mAb20.1 | DIQMNQSPSSLSASLGDTITITCHASQNINVWLSWYQ |
| | | | QKPGNIPKLLIYKASNLHTGVPSRFSGSGSGTGFTLTI |
| | | | SSLQPEDIATYYCQQGQSYPLTFGGGTKLDIK |

### EXAMPLES

### 1. Methods and Assays of the Disclosure

Methods to characterize BTN3A activating antibodies for use according to the present disclosure

### 1.1 Binding Affinity Assay: Multi-Cycle Kinetic Assay (SPR)

Multi-cycle kinetic analysis can be performed on anti-BTN3A antibodies using a Biacore T200 (serial no. 1909913) instrument running Biacore T200 Evaluation Software V2.0.1 (Uppsala, Sweden).

Purified antibodies are diluted to a concentration of 2 μg/ml in 2% BSA/PBS. At the start of each cycle, each antibody is captured on the Protein A at a density (RL) of ~146.5 RU (theoretical value to obtain an RMax of ~50 RU). Following capture, the surface is allowed to stabilize before injection of the BTN3A1 antigen (Sino Biological cat. no. 15973-H08H). BTN3A1 is titrated in 0.1% BSA/HBS-P+ (running buffer) in a two-fold dilution range from 25 to 0.78 nM. The association phase is monitored for 400 seconds and the dissociation phase for 35 minutes (2100 seconds). Kinetic data is obtained using a flow rate of 50 μl/min to minimize any potential mass transfer effects. Regeneration of the Protein A surface is conducted using two injections of 10 mM glycine-HCL pH 1.5 at the end of each cycle. Two blanks (no BTN3A1) and a repeat of a single concentration of the analyte are performed for each tested antibody to check the stability of the surface and analyte over the kinetic cycles. The signal from the reference channel Fc1 is subtracted from that of Fc2, Fc3 and Fc4 to correct for differences in non-specific binding to a reference surface. Additionally, blank runs are subtracted for each Fc to correct any antigen-independent signal variation, such as drift. Sensorgrams are fitted using a one-to-one binding mathematical model with a global RMax parameter and no bulk signal (Constant RI=0 RU).

### 1.2 Binding Assay by Flow Cytometry on Human PBMCs

BTN3A activating antibodies for use according to the present disclosure may also be characterized for their binding to human PBMCs, isolated from blood of healthy donors.

PBMCs are isolated from buffy coats using Lymphoprep (Axis-shield, Dundee, UK) density centrifugation. PBMCs are then frozen and stored at −80° C. or in liquid nitrogen until required.

100 μl cells at 1×10⁶ cells/ml are transferred to each well of a fresh U-shaped bottom 96-well plate, then the plate is centrifuged and supernatant discarded.

A serial dilution of the antibodies, 0.001 μg/ml to 150 μg/ml is prepared in PBS containing 2 mM EDTA. Human PBMCs are resuspended in 50 μl of the diluted test antibody titration series prepared.

After incubation for 30 minutes at 4° C. in the dark, the plate is centrifuged and washed twice with 150 μl/well of PBS+2 mM EDTA following which the cells are resuspended in 50 μl of a mix composed of goat anti-human antibody (PE labelled) diluted 1/100 and Live/dead neat IR diluted 1/500 in PBS+2 mM EDTA.

After incubation for 15 minutes at 4° C. in the dark, the plate is centrifuged and washed once with 150 μl/well PBS+2 mM EDTA following which the cells are resuspended in 200 μl PBS+2 mM EDTA. Cells are analyzed on a BD LSR Fortessa Cytometer. Data is analyzed using a FlowJo software (Version 10, FlowJo, LLC, Ashland, USA).

The same protocol may be performed on cynomolgus PBMCs and on Daudi Burkitt's lymphoma cell line.

### 1.3 In Vitro Functional Efficacy: γδ-T Cell Degranulation Assay

The assay consists of measuring activating or inhibitory effected of anti-BTN3A antibodies on γδ-T cell degranulation against the Daudi Burkitt's lymphoma cell line (Harly et al., 2012, Blood Vol 120, Number 11, pp 2269-2279). γδ-T cells are expanded from PBMCs of healthy donors by culturing with zoledronic acid (1 μM) and IL2 (200 UI/mL) for 11-13 days. IL2 is added at day 5, day 8 and every 2 days thereafter. The percentage of γδ-T cells is determined at the initiation of culture and assessed for the time of culture by flow cytometry until it reaches at least 80%. Frozen or fresh γδ-T cells are then used in degranulation assays against the Daudi cell line (effector:target (E:T) ratio of 1:1), whereby the cells are co-cultured for 4 hours at 37° C. in presence of 10 μg/mL of the 7.2 and/or 20.1 humanized variants and/or their chimeric versions. Activation by PMA (20 ng/mL) plus lonomycin (1 μg/mL) serves as positive control for γδ-T cell degranulation, and medium alone as negative control. At the end of the 4 hours co-incubation, cells are analyzed by flow cytometry to evaluate the percentage of γδ-T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

The same protocol may be performed using AML blasts isolated from patients as target cells, in place of Daudi cells.

### 1.4 In Vitro Functional Efficacy: Vγ9Vδ2 T Cell Activation in PBMC

The assay consists of measuring the activating effect of BTN3A activating antibodies on Vγ9Vδ2 T cells in PBMC. Human PBMCs are isolated by Ficoll density gradient centrifugation of peripheral blood (EDTA-buffy coats or heparinized whole blood). When using whole blood, RBC are depleted using 1×RBC Lysis Buffer (eBioscience) for 10 minutes at room temperature and then washed with PBS+1% FBS.

PBMCs or RBC-depleted cells are cultured in RPMI 1640 containing 10% FBS, 1% Penicillin/Streptomycin at 1.5 to 3×106 cells/mL, with increasing concentrations of BTN3A activating antibodies (dose range 0.00001 to 100 μg/mL) at 37° C., 5% CO2 in a volume of 200 μL in 96 round-bottom well plates. Activation status is monitored after two days of culture by flow cytometry analysis of activation marker surface expression. Cells are washed in in PBS+2% FBS and 2 mM EDTA (FACS buffer). Cells were centrifuged at 1800 rpm for 5 minutes and then incubated 10 minutes at room temperature (RT) with 10 μL of FcR blocking reagent (Miltenyi Biotec) before adding 30 to 50 μL of the appropriate antibody mix prepared in FACS buffer and containing at least fluorescently-conjugated anti-CD3, anti-Vγ9 or Vδ2 TCR and anti-CD69 antibodies. A viability marker (LIVE/DEAD Fixable Dead Cell Stain) was added in all experiments in order to exclude dead cells from the analysis. Cells are incubated 30 minutes at 4° C. and washed 2 times in FACS buffer before fixation in Cytofix Fixation Buffer (BD Bioscience) and flow cytometry analysis. Data is analyzed with flowjo V-10.6 software. Activated Vγ9Vδ2 T cells are defined as CD3+Vδ2+(or Vγ9+ or Vδ2+Vγ9+) CD69+.

### 1.5 In Vitro Vγ9Vδ2 T Cell Expansion

Peripheral blood mononuclear cells (PBMCs) were isolated by Ficoll density gradient centrifugation of peripheral blood obtained from the Etablissement Français du Sang (EFS) Provence Alpes Côte d′Azur (France). To expand Vγ9Vδ2 T cells, 300*10⁶ PBMCs were resuspended at 1.5*10⁶ cells/ml in RPMI1640 supplemented with 10% FBS and 1% sodium pyruvate in 75 cm2 flasks for 10 to 14 days, in presence of rHuIL-2 (200 UI/mL) and aminobisphosphonates (Zoledronate, 1 μM). From day 5, rHuIL-2 was renewed every 2 or 3 days and cells were kept at 1*10⁶ cells/ml. At the end of the expansion phase, the purity of Vγ9Vδ2 T cells was assessed by flow cytometry, and if the number of Vγ9Vδ2 T cells reached 80% of live cells, these cells were then frozen in CryoStor CS10 for future use.

### 1.6 Mouse Model

The selected mouse strain was the highly immunodeficient NSG mice lacking mature T cells, B cells and natural killer (NK) cells and which are also deficient in multiple cytokines signaling pathways, therefore making them the ideal for human cell engraftment. Six to eight-week-old female NSG mice were housed collectively in disposable standard cages in ventilated racks at the TrGET platform facility (Cancer Research Center of Marseille, France). Mice were housed under sterile conditions with sterilized food and water provided ad libitum and were maintained on a 12-h light and 12-h dark cycle and under temperature and humidity control. Cages contained an enriched environment with bedding material.

NSG mice were injected intravenously (iv) via the tail vein at day 0 with 0.2*106 MOLM-14 (CVCL_7916) cells expressing luciferase (luc2) per mouse in a volume of 100 μl. Bioluminescence analysis was performed at day 0 using PhotonIMAGER (Biospace Lab) following addition of endotoxin-free luciferin (30 mg/kg) and mice were randomized in homogeneous groups of 6-8 mice based on the strength of the bioluminescence signal (Table). 3*106 human in vitro-expanded Vγ9Vδ2 T cells and rHuIL-15/IL-15Rα-Fc complexes were iv injected at days 1, 8, 15 and 23. ICT01 or hIgG1S were iv injected at days 1, 5, 8, 11, 15, 18, 23 and 25.

The rHuIL-15/IL-15Rα-Fc complexes were pre-complexed at room temperature (RT) for 30 minutes (0.2 ug rHuIL-15+1.2 ug IL-15Rα-Fc per mice) and mixed with Vγ9Vδ2 T cells and ICT01 or its isotype control (hIgG1S) prior to injection. The final volume for each injection was 200 μl.

Venetoclax was administered to mice via oral gavage (og) on days 1-4, and then 5 days per week for 3 weeks in total. 5-Azacytidine was administered via intraperitoneal (ip) injection on days 1-4 at the same time as Venetoclax treatment. Venetoclax and 5-Azacytidine were given 4-6 hours after Vγ9Vδ2 T cell transfer.

Bioluminescence signal emitted by the MOLM-14 cells was measured at days 0, 7, 14, 21 and 28 after cell injection to follow tumor growth. Daily monitoring of mice for symptoms of disease (significant weight loss, ruffled coat, hunched back, weakness, and reduced mobility) determined the time of killing for injected animals with signs of distress. Survival curves were estimated by the Kaplan-Meier method and compared using the log-rank test.

### 2. Results

### 2.1 ICT01-Mediated Activation of Vγ9Vδ2 T Cells Increases Resistance to Inhibitors of Bcl-2 Family Members.

The effect of ICT01 on the survival of Vγ9Vδ2 T cells upon treatment with Bcl-2 family member inhibitors was assessed in Peripheral Blood Mononuclear Cells from Healthy Donors (HD-PBMC) using the apoptotic marker Caspase3/7 and a dead cell marker in flow cytometry analysis.

Peripheral Blood Mononuclear Cells (PBMC) were isolated by Ficoll density gradient centrifugation of peripheral blood (EDTA-buffy coats) from healthy donors (HD, n=4-6), cultured in PBMC medium (RPMI 1640 Medium supplemented with 10% Fetal Bovine Serum (FBS), 1% Penicillin/Streptomycin and 1 mM Sodium Pyruvate) and treated with ICT01 or its isotype control (hIgG1S) used at 1 μg/mL in the presence or absence of increasing concentrations of Venetoclax (0-0.1 μM), 5-Azacytidine (0-0.4 μM) or the combination, or ABT-737 (0-1.875 μM), Navitoclax (0-1.875 μM) or MIK665 (0-1.875 μM). Cells were harvested on day 2, centrifuged at 1800 rpm for 5 minutes and then incubated 10 minutes at room temperature (RT) with 25 μl of FcR blocking reagent (Miltenyi Biotec) before adding 25 μL of the following antibody mix prepared in in PBS+2% FBS+2 mM EDTA (FACS buffer): anti-CD4-BUV395, CD56-BV421, Vd2-PE, CD3-PE-CF594 and CD8-A700 antibodies for the identification of immune subsets, anti-CD25-BV650 and CD69-APC antibodies were added to monitor activation status of Vγ9Vδ2 T cells, and a viability marker (LIVE/DEAD™ Fixable Dead Cell Stain) was added to distinguish live from dead cells. Cells were incubated 20 minutes at room temperature (RT) and washed 2 times with FACS buffer. A Caspase3/7 Green detection reagent (ThermoFisher) was added to determine apoptosis and cells were incubated for 30 minutes at RT before flow cytometry analysis.

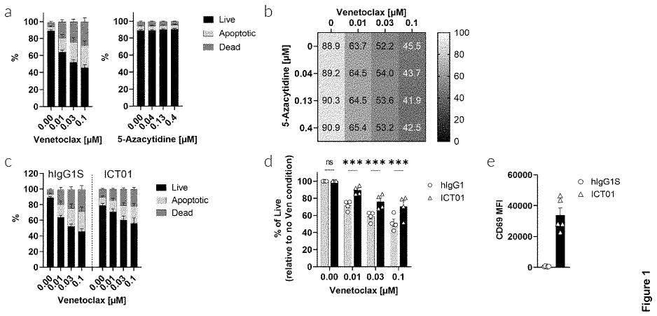

While 5-Azacytidine had no effect on the survival of Vγ9Vδ2 T cells, treatment with Venetoclax led to apoptosis and death of Vγ9Vδ2 T cells in a concentration dependent manner (reduction of live cells by ~29% with 0.01 μM Venetoclax, up to ~49% with 0.1 μM Venetoclax compared to no Venetoclax condition) (FIG. 1a, b). In contrast, simultaneous treatment with ICT01 significantly decreased cell death induced by Venetoclax (reduced survival by ~11% with 0.01 μM Venetoclax and ~30% with 0.1 μM Venetoclax compared to no Venetoclax condition) (FIG. 1c, d). As expected, ICT01 induced specific activation of Vγ9Vδ2 T cells as shown by increased expression of CD69 and CD25 after 2 days of treatment, compared to hIgG1S (FIG. 1e and data not shown). This indicates that activation of Vγ9Vδ2 T cells by ICT01 partially protects them from Venetoclax induced cell death.

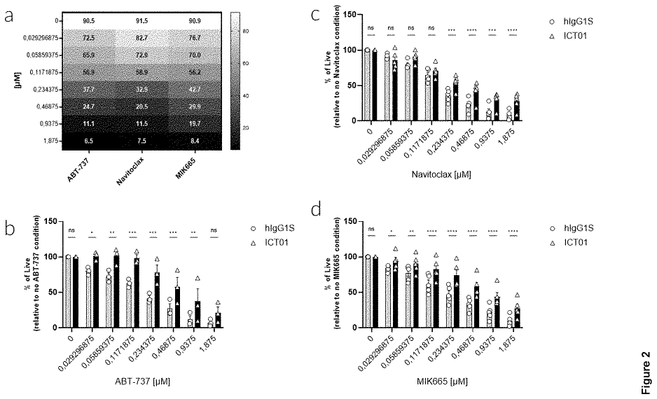

As described for Venetoclax, treatment with other Bcl-2 family member inhibitors induced apoptosis and death of Vγ9Vδ2 T cells in a concentration dependent manner with EC₅₀ of 0.24 μM, 0.16 μM and 0.24 μM for ABT-737, Navitoclax and MIK665 respectively (FIG. 2a). When PBMC were simultaneously treated with ICT01, a significant decrease of Vγ9Vδ2 T cell death induced by Bcl-2 family member inhibitors was observed. EC₅₀ of Vγ9Vδ2 T cell death increased from 0.23 μM in presence of isotype control to 0.46 μM in presence of ICT01 for ABT-737 (FIG. 2b), from 0.16 μM in presence of isotype control to 0.20 μM in presence of ICT01 for Navitoclax (FIG. 2c), and from 0.24 μM in presence of isotype control to 0.78 μM in presence of ICT01 for MIK665 (FIG. 2d).

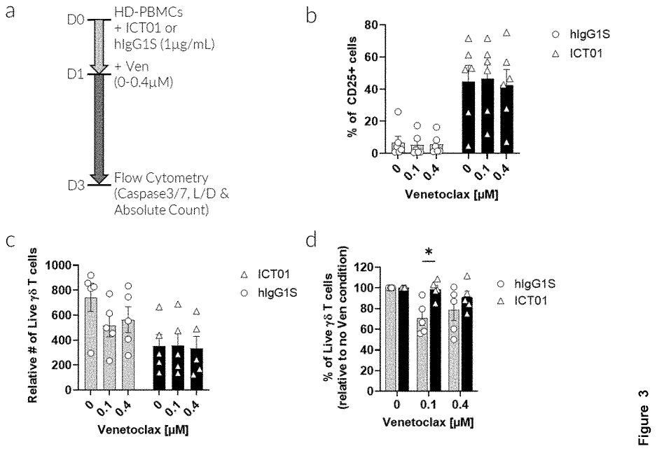

In a similar experiment, freshly isolated PBMC were stimulated with ICT01 or its isotype control hIgG1S for 1 day prior to a 2 day-treatment with Venetoclax (FIG. 3a). The number of live Vγ9Vδ2 T cells was assessed by flow cytometry at the end of the incubation time. The results showed that activation with ICT01 (confirmed by increased surface expression of CD25 (FIG. 3b) significantly reduced Venetoclax induced cell death of Vγ9Vδ2 T cells (Figure c, d).

Altogether, these results indicate that in vitro activation of Vγ9Vδ2 T cells by ICT01 could protect Vγ9Vδ2 T cells from cell death induced by Venetoclax and other inhibitors of the Bcl-2 family members. This would provide an advantage to monotherapy as numbers of Vγ9Vδ2 T cells, which are important in anti-cancer immunity, could act in concert with Venetoclax (or other Bcl-2 family member inhibitors) to kill AML blasts.

### 2.2 Venetoclax and 5-Azacytidine do not Affect ICT01-Induced Activation and Proliferation of Vγ9Vδ2 T Cells

ICT01 has been shown to induce activation and proliferation of resting Vγ9Vδ2 T cells. To further assess the effect of Venetoclax and 5-Azacytidine on Vγ9Vδ2 T cells, ICT01-induced phenotypic activation, IFNγ production and proliferation were measured in healthy donor-derived PBMC cultured with or without Venetoclax, 5-Azacytidine or the combination.

ICT01-induced phenotypic activation of Vγ9Vδ2 T cells is indicated by the upregulation of CD25. Therefore, human PBMC from healthy donors (HD, n=6) were cultured with ICT01 or its isotype control (hIgG1S) used at 1 μg/mL in the presence or absence of increasing concentrations of Venetoclax (0-0.1 μM), 5-Azacytidine (0-0.4 μM) or the combination in PBMC medium (RPMI 1640 Medium supplemented with 10% FBS, 1% Penicillin/Streptomycin and 1 mM Sodium Pyruvate). Cells were harvested on day 4, centrifuged at 1800 rpm for 5 minutes and then incubated 10 minutes at room temperature (RT) with 25 μl of FcR blocking reagent (Miltenyi Biotec) before adding 25 μL of the following antibody mix prepared in PBS+2% FBS+2 mM EDTA (FACS buffer): anti-CD4-BV650, CD56-FITC, Vd2-PE, CD3-PE-CF594 and CD8-A700 antibodies for the identification of immune subsets, an anti-CD25-BV650 antibody was added to monitor activation status, and a viability marker (LIVE/DEAD™ Fixable Dead Cell Stain) was added to distinguish live from dead cells. Cells were incubated 20 minutes at 4° C. and washed 2 times in FACS buffer before flow cytometry analysis.

5-Azacytidine and Venetoclax had no effect on the ICT01-induced upregulation of CD25 by Vγ9Vδ2 T cells and thus no effect on their phenotypic activation (Figure a).

In a similar experiment, freshly isolated healthy donor-derived PBMC were treated with increasing concentrations of Venetoclax (0-0.1 μM), 5-Azacytidine (0-0.4 μM) or the combination for one day prior to stimulation with ICT01 or its isotype control hIgG1S for two days. Again, activation of Vγ9Vδ2T cells by ICT01 was unaffected by Venetoclax and 5-Azacytidine treatment, as indicated by the similar induction of CD25 expression in all conditions tested (Figure b).

Vγ9Vδ2 T cell functions are tightly regulated by activating (NKG2D and DNAM-1) and inhibitory receptors (PD-1, NKG2A, BTLA, TIM3). To assess the impact of Venetoclax and 5-Azacytidine treatment on the expression of these receptors by Vγ9Vδ2 T cells, frozen human PBMC from healthy donors (HD, n=6) were thawed and treated with ICT01 or its isotype control (hIgG1S) used at 1 μg/mL in the presence or absence of increasing concentrations of Venetoclax (0-0.1 μM), 5-Azacytidine (0-0.4 μM) or the combination in PBMC medium (RPMI 1640 Medium supplemented with 10% FBS, 1% Penicillin/Streptomycin, 1 mM Sodium Pyruvate and 50 IU/mL IL2). Cells were harvested on day 5, centrifuged at 1800 rpm for 5 minutes and then incubated 10 minutes at RT with 25 μl of FcR blocking reagent (Miltenyi Biotec) before adding 25 μL of the following antibody mix prepared in PBS+2% FBS+2 mM EDTA (FACS buffer): anti-CD4-BUV395, Vδ2-PE, CD3-PE-CF594 and CD8-A700 antibodies for the identification of immune subsets, anti-BTLA-BV421, PD1-BV650, DNAM-1-BV785, TIM3-BB515, NKG2A-PE-Vio770 and NKG2D-APC antibodies for the detection of inhibitory and activating receptors, and a viability marker (LIVE/DEAD™ Fixable Dead Cell Stain) was added to distinguish live from dead cells. Cells were incubated 20 minutes at 4° C. and washed 2 times in FACS buffer before flow cytometry analysis.

ICT01 induced an increased expression of all analyzed activating and inhibitory receptors on Vγ9Vδ2 T cells by day 5 of culture, which was unaffected by treatment with Venetoclax and 5-Azacytidine (data not shown).

To evaluate the effect of Venetoclax and 5-Azacytidine on cytokine production, freshly isolated human PBMC from healthy donors (HD, n=6) were cultured in PBMC medium (RPMI 1640 Medium supplemented with 10% Fetal Bovine Serum (FBS), 1% Penicillin/Streptomycin and 1 mM Sodium Pyruvate) and treated with ICT01 or its isotype control (hIgG1S) used at 1 μg/mL in the presence or absence of increasing concentrations of Venetoclax (0-0.1 μM), 5-Azacytidine (0-0.4 μM) or the combination. Half of the cells were additionally treated with 50 IU/mL IL2. Golgi Stop was added after 6-8 hours of co-culture and cells were harvested 16 h later, centrifuged at 1800 rpm for 5 minutes and then incubated 10 minutes at room temperature (RT) with 25 μl of FcR blocking reagent (Miltenyi Biotec) before adding 25 μL of the following antibody mix prepared in PBS+2% FBS+2 mM EDTA (FACS buffer): anti-CD4-BV650, Vγ9-FITC, CD3-PE-CF594, CD56-PE-Vio770 and CD8-AF700 antibodies for the identification of immune subsets, and a viability marker (LIVE/DEAD™ Fixable Dead Cell Stain) was added to distinguish live from dead cells. After 20 minutes, 100 μL of CytoFix solution (BD Bioscience) was added, and cells were incubated for 20 minutes at 4° C. 100 μL of Perm/Wash (BD Bioscience) were added, cells centrifuged and washed once in 200 μL Perm/Wash. After washing, cells were stained with anti-IFNγ-APC antibody for 30 minutes at 4° C., washed twice with Perm/Wash, and analyzed by flow cytometry.

Treatment with Venetoclax or 5-Azacytidine alone had no effect on ICT01-induced IFNγ production by Vγ9Vδ2 T cells independent of IL2 (data not shown). Treatment with both Venetoclax and 5-Azacytidine in combination reduced IFNγ production by Vγ9Vδ2 T cells from 30.4 to 25.2% when activated with ICT01 alone, which was not observed in presence of IL2 (89.1% without treatment and 91.7% with Venetoclax and 5-Azacytidine in combination, data not shown).

Finally, freshly isolated human PBMC from healthy donors (HD, n=6) were labeled with CellTrace Violet stain (CTV, Invitrogen) to determine cell proliferation upon treatment with ICT01 or its isotype control (hIgG1S) used at 1 μg/mL in the presence or absence of increasing concentrations of Venetoclax (0-0.1 μM), 5-Azacytidine (0-0.4 μM) or the combination in PBMC medium (RPMI 1640 Medium supplemented with 10% FBS, 1% Penicillin/Streptomycin and 1 mM Sodium Pyruvate). Half of the cells were additionally treated with 50 IU/mL IL2. Cells were harvested on day 4, centrifuged at 1800 rpm for 5 minutes and then incubated 10 minutes at RT with 25 μl of FcR blocking reagent (Miltenyi Biotec) before adding 25 μL of the following antibody mix prepared in PBS+2% FBS+2 mM EDTA (FACS buffer): anti-CD4-BV650, CD56-FITC, Vδ2-PE, CD3-PE-CF594 and CD8-A700 antibodies for the identification of immune subsets, and a viability marker (LIVE/DEAD™ Fixable Dead Cell Stain) was added to distinguish live from dead cells. Cells were incubated 20 minutes at 4° C. and washed 2 times in FACS buffer before flow cytometry analysis.

Venetoclax increased ICT01-induced proliferation of Vγ9Vδ2 T cells by ~10% that was not observed for 5-Azacytidine alone or with both agents in combination (Figure). ICT01 and IL2-induced proliferation of Vγ9Vδ2 T cells was unaffected by Venetoclax and 5-Azacytidine alone or in combination (Figured).

Taken together, data demonstrate that Venetoclax and 5-Azacytidine treatment as single agents or in combination do not interfere with ICT01-induced activation or proliferation of Vγ9Vδ2 T cells. Therefore, a combination of treatments would protect Vγ9Vδ2 T cells from Venetoclax induced cell death, induce their proliferation and activation, and potentially increased numbers of cytotoxic Vγ9Vδ2 T cells in patients.

2.3 Combined Treatment of Vγ9Vδ2 T Cells with Venetoclax, HMA and BTN3A Activating Antibodies Increases AML Killing

The effect of Venetoclax and 5-Azacytidine on anti-BTN3A antibodies-induced killing of AML cell lines by Vγ9Vδ2 T cells was assessed by measuring the relative number of surviving target cells in a HD-PBMC-AML cell co-culture using flow cytometry.

First, to assess the sensitivity of KG1a AML cell line to Venetoclax and 5-Azacytidine treatment in vitro, cells were treated with increasing concentrations of Venetoclax (0-5 μM), 5-Azacytidine (0-10 μM) or the combination for 2 days in cell culture medium (alpha-MEM+Glutamax supplemented with 20% Fetal Bovine Serum (FBS) and 1 mM Sodium Pyruvate). On day 2, cells were centrifuged at 1800 rpm for 5 minutes and the supernatant discarded. Cells were lysed with Cell Titer Glo reagent (Promega), and ATP amount was monitored as a measure of living cells using luminescence.

KG1a AML cells showed EC₅₀ values of 1.82 μM for Venetoclax and 4.72 μM for 5-Azacytidine with a mean survival of 20.2% with Venetoclax at 5 μM and 47.2% with 5-Azacytidine at 10 μM. The combination of Venetoclax at 5 μM and 5-Azacytidine at 10 μM led to a mean survival of KG1a AML cells of 0.9%, demonstrating a synergistic effect of Venetoclax and 5-Azacytidine treatment (FIG. a).

Then, KG1a AML cells were stained with CellTrace CFSE Proliferation dye (ThermoFisher) to allow for specific detection by flow cytometry, counted, put into a co-culture with thawed HD-PBMC and treated with the anti-BTN3A mAbs ICT01 or m20.1 or their respective isotype controls (hIgG1S or mIgG1) in PBMC medium (RPMI 1640 Medium supplemented with 10% Fetal Bovine Serum (FBS) and 1 mM Sodium Pyruvate). On day one of culture, increasing concentrations of Venetoclax (0-0.4 μM), 5-Azacytidine (0-0.4 μM) or the combination were added. Cells were harvested on day 3 of co-culture, centrifuged at 1800 rpm for 5 minutes and then incubated 10 minutes at room temperature (RT) with 25 μl of FcR blocking reagent (Miltenyi Biotec) before adding 25 μL of the following antibody mix prepared in PBS+2% FBS+2 mM EDTA (FACS buffer): anti-CD56-BV421, CD19-BV421, CD14-BV785, Tyb-PE and CD3-AF700 antibodies for the identification of immune subsets, and a viability marker (LIVE/DEAD™ Fixable Dead Cell Stain) was added to distinguish live and dead cells. Cells were incubated 20 minutes at room temperature, washed once in FACS buffer and once in AnnexinV staining buffer. An AnnexinV-APC probe mixed with CountBright Absolute Counting Beads (Invitrogen) was added to determine apoptosis and relative cell numbers, followed by 15 minutes incubation at RT before flow cytometry analysis.

In this assay, KG1a were rather resistant to BTN3A activating antibody-mediated killing by Vγ9Vδ2 T cells (average reduction of live KG1a cell numbers by ~20% with ICT01 compared to hIgG1S conditions with large variability within PBMC donors) (FIG. B). Treatment with 5-Azacytidine had no effect on KG1a survival in hIgG1S conditions (Figured). In contrast, in hIgG1S-treatment conditions, Venetoclax reduced KG1a relative numbers by ~28% at the highest concentration and by 44% in combination with 5-Azacytidine. Upon activation with ICT01, treatment with Venetoclax and 5-Azacytidine alone had slight increasing effects on Vγ9Vδ2 T cell killing activity compared to hIgG1S conditions. However, Venetoclax and 5-Azacytidine in combination significantly reduced the relative number of live KG1a cells to ~46.5% compared to hIgG1S conditions (p<0.05), and to ~32.9% compared to no Venetoclax and 5-Azacytidine treatment, demonstrating a synergistic effect of the combined treatments.

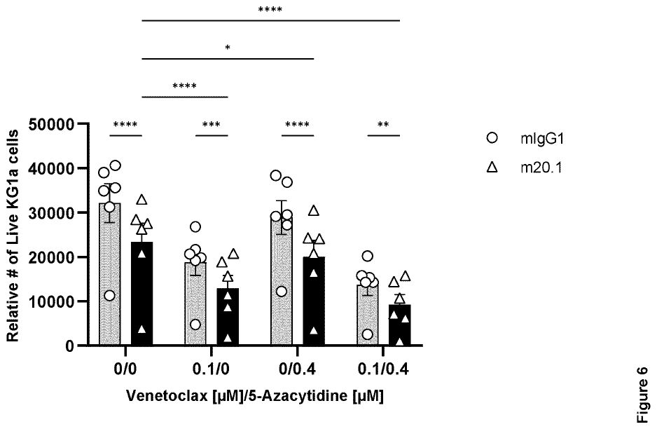

Similar results were obtained with the mAb 20.1 BTN3A activating antibody (FIG. 6). In mIgG1 conditions, while treatment with 5-Azacytidine had no effect on KG1a survival, Venetoclax used at 0.1 μM reduced KG1a relative numbers by 41% and by 57% when combined with 0.4 μM of 5-Azacytidine. In addition, the combination of 20.1 mAb with Venetoclax and 5-Azacytidine significantly reduced the relative number of live KG1a cells as compared to either m20.1 used alone or Venetoclax and 5-Azacytidine used with the isotype control with ~67% reduction of live KG1a cells as compared to control condition (FIG. 6).

Taken together, these data demonstrate that activation of PBMC from healthy donors with ICT01 increases Vγ9Vδ2 T cell survival towards Venetoclax induced cell death and that Venetoclax and 5-Azacytidine treatment does not impact activation or proliferation of Vγ9Vδ2 T cells in HD-derived PBMC in vitro. In addition, combination of BTN3A antibodies-activated Vγ9Vδ2 T cells in HD-PBMC with Venetoclax and 5-Azacytidine treatment significantly reduced the survival of the Vγ9Vδ2 T cell-resistant AML cell line KG1a.

2.4 Combined Treatment with Venetoclax, HMA and ICT01 Increases Vγ9Vδ2 T Cell-Mediated Killing of Burkitt Lymphoma and Chronic-B-Cell Leukemia Cell Lines

The effect of Venetoclax and 5-Azacytidine on BTN3A activating antibody-induced killing of Burkitt Lymphoma and Chronic-B-cell leukemia cell lines by Vγ9Vδ2 T cells was assessed by measuring the relative number of surviving target cells in a HD-PBMC-Target cell co-culture using flow cytometry.

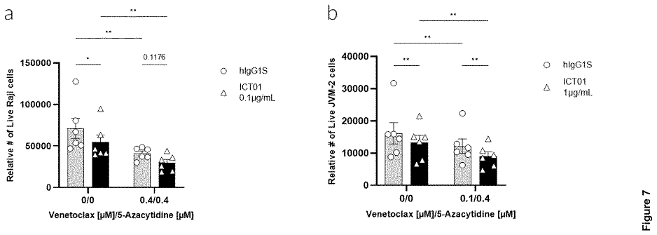

In presence of the BTN3A activating antibody ICT01, co-culture of Raji (Burkitts Lymphoma) or JVM-2 (Chronic B-cell leukemia) with HD-PBMC for 48 hrs induced ~22% and ~16% decrease of live cells respectively with large variability within PBMC donors (FIG. 7a, b). In hIgG1S-treatment conditions, Venetoclax combined with 5-Azacytidine at indicated concentration reduced the relative numbers of targets by ~37% and ~32% for Raji and JVM2 respectively as compared to hIgG1S alone condition (control). Importantly, the combination of ICT01, Venetoclax and 5-Azacytidine still reduced the relative number of live target cells after 48 hrs of co-culture with ~55% and ~46% decrease of live target cell number as compared the control condition demonstrating the benefit combining BTN3A activating antibody such as ICT01 with Venetoclax and 5-Azacytidine for killing of Burkitt Lymphoma and Chronic-B-cell leukemia cell lines.

Taken together, these data suggest that combination of BTN3A activating antibody-activated Vγ9Vδ2 T cells in HD-PBMC with Venetoclax and 5-Azacytidine treatment significantly reduced the survival of several hematological cancer cell lines and provides an advantage for the treatment of hematological malignancies.

2.5 Combined Treatment of ICT01-Activated Vγ9Vδ2 T Cells with Bcl-2 Family Member Inhibitors Increases AML Killing

The effect of the three Bcl-2 family member inhibitors ABT-737, Navitoclax and MIK665, on ICT01-induced killing of AML cell lines by Vγ9Vδ2 T cells was assessed by measuring the relative number of surviving target cells in a HD-PBMC-AML cells co-culture using flow cytometry.

For assays using ABT-737 or Navitoclax, KG1a cells were used. However, this cell line does not express MCL-1 and is consequently completely resistant to MIK665 treatment. Therefore, MOLM14 cells, which express MCL-1, were used in killing assays assessing the activity of ICT01 combined with MIK665.

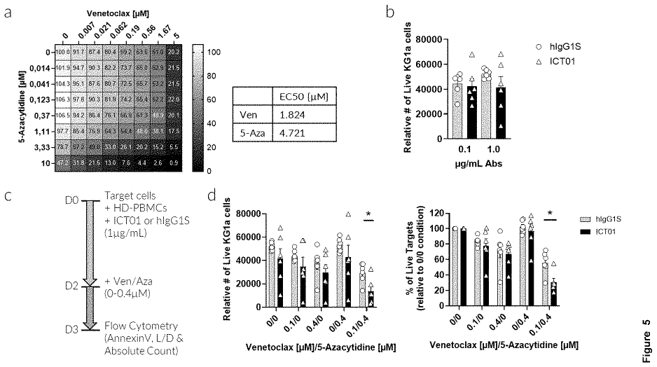

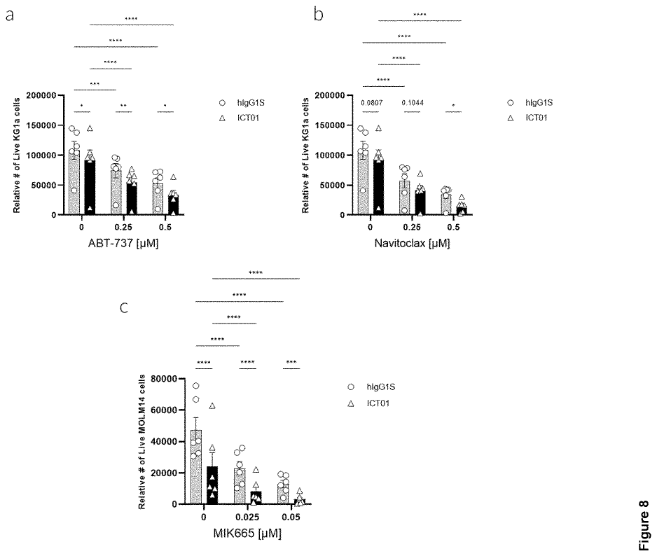

Killing of KG1a co-cultured with HD-PBMC in presence of ICT01, ABT-737 or the combination (FIG. 8a). In this experiment ICT01 induced ~21% of KG1a cell death as compared to control condition (similar to what was obtained previously (FIG. 5b)). Treatment with ABT-737 alone at 0.25 μM and 0.5 μM induced respectively ~34% and ~54% of KG1a cell death. When the co-cultures of KG1a and HD-PBMC were treated with the combination of ICT01 and ABT-737, the number of living KG1a cells was reduced by ~53% and ~71% (for ABT-737 used at 0.25 μM and 0.5 μM respectively) as compared to control demonstrating the benefit of this combination for AML cell line killing (FIG. 8a).

Killing of KG1a co-cultured with HD-PBMC in presence of ICT01, Navitoclax or the combination (FIG. 8b). Treatment with Navitoclax alone at 0.25 μM and 0.5 μM induced respectively ~51% and ~71% of KG1a cell death. When the co-cultures of KG1a and HD-PBMC were treated with the combination of ICT01 and Navitoclax, the number of living KG1a cells was reduced by ~65% and ~88% (for Navitoclax used at 0.25 μM and 0.5 μM respectively) as compared to control demonstrating the benefit of this combination for AML cell line killing (FIG. 8b).

Killing of MOLM14 co-cultured with HD-PBMC in presence of ICT01, MIK665 or the combination (FIG. 8c). In this experiment ICT01 induced ~56% of MOLM14 cell death as compared to control condition. Treatment with MIK665 alone at 0.025 μM and 0.05 μM induced respectively ~52% and ~72% of KG1a cell death. When the co-cultures of MOLM14 and HD-PBMC were treated with the combination of ICT01 and MIK665, the number of living MOLM14 cells was reduced by ~85% and ~93% (for MI-665 used at 0.025 μM and 0.05 μM respectively) as compare to control demonstrating the benefit of this combination for AML cell line killing (FIG. 8c).

Taken together, these data demonstrate that the combination of ICT01-activated Vγ9Vδ2 T cells in HD-PBMC with Bcl-2 family member inhibitors treatment, significantly reduced the survival of AML cell lines confirming the potential of using ICT01 in combination with Bcl-2 family member inhibitors to treat hematologic malignancies.

2.6 ICT01 Combined with Venetoclax and 5-Azacytidine Significantly Prolongs Survival of MOLM-14 Engrafted NSG Mice

The in vivo efficacy of ICT01 in combination with Venetoclax and 5-Azacytidine against MOLM-14, an AraC resistant human AML derived cell line, was evaluated in a xenograft model using NSG mice transplanted with the human tumor cell line, and adoptively transferred with human Vγ9Vδ2 T cells. The goal of this study was to assess the effect of repeated iv injections of ICT01-activated human Vγ9Vδ2 T cells in combination with Ven/Aza on tumor growth and on mice survival.

NSG mice were injected at day 0 with MOLM-14 and randomized in homogeneous groups based on the strength of the bioluminescence signal. The different experimental groups are summarized in Table.

TABLE 5
MOLM-14 mouse model: group description

| | Number of | |
|---|---|---|
| Group | animals | Treatment |
| 1 | 7 | PBS |
| 2 | 8 | Venetoclax (40 mg/kg) + Azacytidine (2 mg/kg) |
| 3 | 6 | Vγ9Vδ2 T cells (3*10⁶ cells) + rHuIL-15/IL-15Rα-Fc + hIgG1S |
| | | (1 mg/kg) |
| 4 | 6 | Vγ9Vδ2 T cells (3x*10⁶ cells) + rHuIL-15/IL-15Rα-Fc + ICT01 |
| | | (1 mg/kg) |
| 5 | 6 | Vγ9Vδ2 T cells (3*10⁶ cells) + rHuIL-15/IL-15Rα-Fc + hIgG1S |
| | | (1 mg/kg) + Venetoclax (40 mg/kg) + Azacytidine (2 mg/kg) |
| 6 | 6 | Vγ9Vδ2 T cells (3*10⁶ cells) + rHuIL-15/IL-15Rα-Fc + ICT01 |
| | | (1 mg/kg) + Venetoclax (40 mg/kg) + Azacytidine (2 mg/kg) |

As shown in Table, there was no basal control of MOLM-14 in vivo growth by human Vγ9Vδ2 T cells injected with irrelevant control isotype antibody (hIgG1S) (Group 1 vs Group 3). In contrast, as shown by lower bioluminescence signals and median survival (Table 6 and Figure), tumour growth was significantly delayed when human Vγ9Vδ2 T cells were administered with the BTN3A activating mAb ICT01 (Median survival of 24 and 28 days for Group 3 and Group 4 respectively) or in mice treated with Venetoclax and 5-Azacytidine (Median survival of 24 and 29 days for Group 1 and Group 2 respectively). Combination of Venetoclax and 5-Azacytidine treatment with transfer of non-activated human Vγ9Vδ2 T cells prolonged median survival of mice to 32.5 days (Group 2 and 3 vs Group 5) indicating that Venetoclax and 5-Azacytidine treatment renders cells more susceptible to killing by human Vγ9Vδ2 T cells (Table).

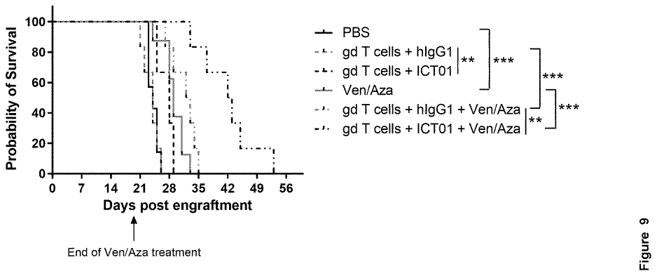

Importantly, treatment of mice with ICT01, human Vγ9Vδ2 T cells, Venetoclax and 5-Azacytidine strikingly improved median survival to 42.5 days (Group 6) (FIG. 9).

These data confirm that combination of B3TN3A activating antibody such as ICT01 with Bcl2 family inhibitors such as Venetoclax, and 5-Azacytidine could be a promising approach for the treatment of AML patients.

TABLE 6
MOLM-14 mouse model: Mean bioluminescence measurement per group
at day 0, 7, 14, 21 and 28 after tumor cell engraftment.

| | | Day 0 | Day 7 | Day 14 | Day 21 | Day 28 |
|---|---|---|---|---|---|---|
| Group 1 | Mean | 13511 | 57370 | 18157143 | 255250000 | Non Applicable |
| PBS | (n = 7) | | | | | |
| | SD | 6786 | 49656 | 10418430 | 42311346 | Non Applicable |
| | SEM | 2565 | 18768 | 3937797 | 21155673 | Non Applicable |
| Group 2 | Mean | 12689 | 6090 | 637750 | 26611250 | 53466667 |
| Ven/Aza | (n = 8) | | | | | |
| | SD | 7980 | 2121 | 461839 | 15863431 | 63784408 |
| | SEM | 2821 | 750 | 163285 | 5608570 | 26039875 |
| Group 3 | Mean | 14960 | 31708 | 10378333 | 264250000 | Non Applicable |
| Vγ9Vδ2 T cells + | (n = 6) | | | | | |
| hIgG1S | SD | 4550 | 16033 | 8824850 | 87887712 | Non Applicable |
| | SEM | 1858 | 6545 | 3602730 | 43943856 | Non Applicable |
| Group 4 | Mean | 15967 | 18452 | 2275500 | 36636667 | 141333333 |
| Vγ9Vδ2 T cells + | (n = 6) | | | | | |
| ICT01 | SD | 7121 | 18566 | 2699914 | 35198478 | 40771722 |
| | SEM | 2907 | 7580 | 1102235 | 14369719 | 23539565 |
| Group 5 | Mean | 15384 | 5505 | 181083 | 13166667 | 39780000 |
| Vγ9Vδ2 T cells + | (n = 6) | | | | | |
| hIgG1S + | SD | 6492 | 2690 | 79402 | 17040405 | 16640222 |
| Ven/Aza | SEM | 2650 | 1098 | 32416 | 6956716 | 7441734 |
| Group 6 | Mean | 15076 | 2615 | 26817 | 1023833 | 4794167 |
| Vγ9Vδ2 T cells + | (n = 6) | | | | | |
| ICT01 + | SD | 6099 | 1540 | 10929 | 793413 | 5125556 |
| Ven/Aza | SEM | 2490 | 629 | 4462 | 323909 | 2092499 |

TABLE 7
MOLM-14 mouse model: animal survival.
Median survival for each group.

| | Median |
|---|---|
| | Survival |
| Groups | (Days) |
| Group 1: PBS (n = 7) | 24 |
| Group 2: Ven/Aza (n = 8) | 29 |
| Group 3: Vγ9Vδ2 T cells + hIgG1S (n = 6) | 24 |
| Group 4: Vγ9Vδ2 T cells + ICT01 (n = 6) | 28 |
| Group 5: Vγ9Vδ2 T cells + hIgG1S + Ven/Aza (n = 6) | 32.5 |
| Group 6: Vγ9Vδ2 T cells + ICT01 + Ven/Aza (n = 6) | 42.5 |

1. A method of treating cancer, comprising administering a BTN3A activating antibody to a subject in need thereof wherein a therapeutically efficient amount of said BTN3A activating antibody is administered to said subject, simultaneously, sequentially or separately, with a therapeutically efficient amount of a Bcl2 inhibitor compound, optionally further in combination with a hypomethylating agent.

2. The method according to claim 1, wherein said Bcl2 inhibitor is Venetoclax.

3. The method according to claim 1, wherein said BTN3A activating antibody comprises (a) a variable heavy chain (VH) polypeptide comprising an amino acid sequence that is at least about 95%, 96%, 97%, 98%, 99% or 100% identical to the amino acid sequence of SEQ ID NO: 1, and (b) a variable light chain (VL) polypeptide comprising an amino acid sequence that is at least about 95%, 96%, 97%, 98%, 99% or 100% identical to of SEQ ID NO: 2;

4. The method according to claim 1, wherein the anti-BTN3A antibody is an antibody comprising a heavy chain of SEQ ID NO: 4 and a light chain of SEQ ID NO: 6.

5. The method according to claim 1, wherein said BTN3A activating antibody is administered in combination simultaneously, sequentially or separately with Venetoclax, further in combination with Azacytidine or Decitabine.

6. The method according to claim 1, wherein said cancer is a hematological malignancy, for example acute myeloid leukemia.

7. The method according to claim 1, wherein said cancer is acute myeloid leukemia, and wherein said BTN3A activating antibody is an antibody comprising a heavy chain of SEQ ID NO: 4 and a light chain of SEQ ID NO: 6, and said BTN3A activating antibody is administered in combination simultaneously, sequentially or separately with Venetoclax, further in combination with Azacytidine or Decitabine.

8. The method according to claim 1, wherein said subject is not eligible for intensive chemotherapy.

9. The method according to claim 1, wherein said BTN3A activating antibody is administered once every three weeks, or once every four weeks.

10. The method according to claim 1, wherein the BTN3A activating antibody is administered intravenously.

11. The method according to claim 1, wherein the BTN3A activating antibody is administered at a unit dose of about 7 to about 200 mg, e.g., 75 mg, for example once every 21 days for 1 to 22 cycles.

12. The method according to claim 1, wherein Venetoclax is administered orally, once a day.

13. The method according to claim 1, wherein Venetoclax is administered orally at a unit dose of about 50 mg to about 500 mg.

14. The method according to claim 1, wherein the hypomethylating agent comprises Azacitidine.

15. The method according to claim 1, wherein the hypomethylating agent is administered at a dose of about 50 mg/m² to about 100 mg/m².
